(19) United States
(12) Patent Application Publication
(10) Pub. No.: US 2026/0354902 A1
(43) Pub. Date: Nov. 12, 2026

(54) SYSTEM AND METHOD FOR HOLOGRAPHIC AUGMENTED REALITY PLANNING AND GUIDANCE IN MEDICAL PROCEDURES UTILIZING A ROBOTIC OR MANUAL ARM

(71) Applicant: MEDIVIEW XR, INC, Cleveland, OH (US)

(72) Inventors: Aydan Thomas Hanlon, Green, OH (US); Ross Daniel Hinrichsen, Plymouth, MN (US); Seyedsina Shirinpour, Minneapolis, MN (US); Jeff Harold Yanof, Solon, OH (US); Peter Nicholas Braido, Wyoming, MN (US); Mina S. Fahim, New Brighton, MN (US); Adam E. Rakestraw, Findlay, OH (US); Gregory Pak Lung Wong, Folsom, CA (US); Cameron Nicholas Mar, Cleveland Heights, OH (US)

(21) Appl. No.: 19/673,279

(22) Filed: May 11, 2026

Related U.S. Application Data
(60) Provisional application No. 63/802,731, filed on May 9, 2025.

(51) Int. Cl. A61B 34/10; A61B 34/00; A61B 34/20; A61B 34/30; A61B 90/00; A61N 7/02

(52) U.S. Cl. CPC A61B 34/10; A61B 34/20; A61B 34/25; A61B 34/30; A61B 90/04; A61B 90/37; A61N 7/02; A61B 2034/107; A61B 2034/2048; A61B 2034/2051; A61B 2034/2055; A61B 2034/2063; A61B 2090/0472; A61B 2090/365; A61B 2090/378

(57) ABSTRACT

A system and method for guiding a medical procedure under holographic augmented reality guidance comprises an augmented reality visualization system including a head-mounted device, a treatment delivery system including a robotic linkage and a therapeutic delivery tool, and a registration tracking system configured to establish and maintain a common coordinate system shared by the augmented reality visualization system and the treatment delivery system throughout localization, planning, and treatment phases of the medical procedure. The augmented reality visualization system displays holographic representations of the therapeutic energy beam, planned therapeutic volumes, and three-dimensional anatomical structures overlaid on the patient through the head-mounted device. Methods for localizing a target tissue, planning a therapeutic procedure, and delivering non-contacting therapeutic energy under holographic augmented reality guidance are also disclosed, including respiratory motion compensation, automated safety interlocks, real-time holographic treatment progress visualization, and post-procedure review of treatment zone coverage.

### CROSS-REFERENCE TO RELATED APPLICATIONS

This application claims the benefit of U.S. Provisional Application No. 63/802,731, filed on May 9, 2025 the disclosure of which is incorporated herein by reference in its entirety.

### FIELD

The present technology relates to medical imaging and therapeutic energy delivery systems and, more particularly, to augmented reality visualization and robotic control or arm-mounted systems for medical procedures.

### INTRODUCTION

This section provides background information related to the present disclosure which is not necessarily prior art.

Non-contacting energy ablation procedures and percutaneous procedures allow for treating patients without invasive surgery. These procedures use focused ultrasound energy delivered from outside the body or use a percutaneously placed needle, probe or other device to precisely target and destroy tissue or deliver other therapies. However, physicians face several challenges when performing these procedures.

Many surgeons lack sufficient training and experience with ultrasound imaging, making it difficult to effectively visualize and locate target tissues during procedures. The planning process remains disjointed, as physicians must mentally correlate pre-operative CT or MRI scans shown on 2D screens with real-time ultrasound imaging. This becomes particularly challenging after the diagnostic ultrasound transducer is moved following initial target acquisition.

During treatment delivery, physicians can struggle to clearly visualize the treatment zone under ultrasound guidance. When using an automated treatment process the user has less control compared to other ablative technologies. With histotripsy maintaining precise targeting becomes difficult once the coupling membrane is applied and the robotic arm is repositioned. Patient anatomy can shift or deform and the water bath must remain parallel to the floor, limiting the movement of the treatment head compared to the diagnostic ultrasound transducer. Additionally, respiratory motion can impact targeting accuracy throughout the procedure.

The integration of pre-operative imaging alongside ultrasound currently has limited utility due to the constraints of 2D displays. Three-dimensional volumetric treatment planning also loses effectiveness when overlaid onto two-dimensional ultrasound views. These visualization and control limitations, combined with the technical complexity of non-contact energy delivery and other percutaneous procedures, create significant barriers to widespread adoption of these promising therapeutic approaches.

Accordingly, there is a continuing need for improved systems and methods that enhance visualization, streamline planning, and provide better control during non-contacting energy ablation procedures and other percutaneous procedures.

### SUMMARY

In concordance with the instant disclosure, improved systems and methods that enhance visualization, streamline planning, and provide better control during non-contacting energy ablation procedures and other percutaneous procedures have surprisingly been discovered.

The present technology includes articles of manufacture, systems, and processes that relate to augmented reality visualization systems, robotic control mechanisms, registration methods, and comprehensive workflow solutions for enhancing target localization, treatment planning, therapeutic delivery, procedural navigation, real-time monitoring, and treatment tracking during non-contacting energy ablation procedures and other percutaneous procedures. The system enables continuous tracking and monitoring throughout all procedural phases while maintaining spatial registration between visualization components and therapeutic delivery systems, whether guided by ultrasound or other imaging modalities.

In one embodiment, a system for guiding a medical procedure comprises an augmented reality visualization system comprising a head-mounted device configured to display a holographic representation, a treatment delivery system comprising a robotic linkage and a therapeutic delivery tool selected from the group consisting of non-contacting energy delivery devices, percutaneous devices, ablation devices, and drug delivery devices, and a registration tracking system including a tracking sensor and configured to establish a common coordinate system shared by the augmented reality visualization system and the treatment delivery system. The registration tracking system is configured to maintain continuous spatial alignment between the augmented reality visualization system and the treatment delivery system throughout localization, planning, and treatment phases of the medical procedure, and the augmented reality visualization system is configured to display a holographic representation of a therapeutic energy beam of the therapeutic delivery tool relative to a tracked position and orientation of the therapeutic delivery tool through the head-mounted device.

In another embodiment, a method for localizing a target tissue under holographic augmented reality guidance using the system described herein comprises establishing registration between the head-mounted device and the treatment delivery system through the registration tracking system to define the common coordinate system, positioning the therapeutic delivery tool using the robotic linkage to locate the target tissue, and stamping and saving a position and orientation of the therapeutic delivery tool in the common coordinate system through the registration tracking system.

In yet another embodiment, a method for planning a non-contacting energy delivery procedure under holographic augmented reality guidance using the system described herein comprises receiving a stamped position and orientation of the therapeutic delivery tool from the registration tracking system, directing the robotic linkage to move the therapeutic delivery tool away from the patient to permit preparation of the therapeutic delivery tool, returning the therapeutic delivery tool to the stamped position, and defining one or more planned therapeutic volumes relative to the target tissue through the augmented reality visualization system.

In a further embodiment, a method for delivering non-contacting therapeutic energy under holographic augmented reality guidance using the system described herein comprises confirming registration between the head-mounted device and the treatment delivery system through the registration tracking system, initiating automated energy delivery through the treatment delivery system wherein the robotic linkage moves the therapeutic delivery tool through a planned energy delivery pattern, and continuously monitoring energy delivery for one or more safety conditions and automatically pausing or disabling energy delivery when a safety condition is triggered.

Further areas of applicability will become apparent from the description provided herein for both non-contacting and percutaneous procedures. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a schematic diagram of a system for holographic augmented reality guidance of a medical procedure, illustrating the augmented reality visualization system, registration tracking system, treatment delivery system, and imaging system.

FIG. 2 is a schematic diagram illustrating registration without image-based anatomy, a first embodiment in which a tracking sensor is mounted on the robotic arm or treatment head for direct tracking, and a second embodiment in which a tracking sensor is mounted on a main unit of the treatment delivery system and the position of the treatment head is determined through kinematic solving.

FIG. 3 is a schematic diagram illustrating registration with image-based anatomy, showing CT or MRI fusion to the patient coordinate space with an optional time-of-flight camera shown as a dashed element for respiratory motion compensation.

FIG. 4 is a schematic diagram illustrating the probe placement algorithm, showing a rotated treatment head and a ranked set of validated placement suggestions output by the algorithm relative to the patient anatomy.

FIG. 5 is a schematic diagram illustrating alternative tracking modalities, showing an optical tracker embodiment and infrared, acoustic, or electromagnetic tracking alternatives for use when the treatment delivery system does not provide an internal coordinate system.

FIG. 6 is a schematic diagram illustrating a radiotherapy application of the system, showing integration with a non-contacting energy delivery device such as a stereotactic radiosurgery system.

FIG. 7 is a flowchart illustrating a method for localizing a target tissue under holographic augmented reality guidance using the system of FIG. 1.

FIG. 8 is a flowchart illustrating a method for planning a non-contacting energy delivery procedure under holographic augmented reality guidance using the system of FIG. 1.

FIG. 9 is a flowchart illustrating a method for delivering non-contacting therapeutic energy under holographic augmented reality guidance using the system of FIG. 1.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1–10, or 2–9, or 3–8, it is also envisioned that Parameter X may have other ranges of values including 1–9, 1–8, 1–3, 1–2, 2–10, 2–8, 2–3, 3–10, 3–9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

As used herein, the terms "interventional device" or "tracked instrument" refer to any medical instrument used during a medical procedure, including but not limited to needles, probes, scalpels, trocars, screwdrivers, forceps, syringes, catheters, ablation probes, and other surgical tools and instruments.

As used herein, the terms "registration device" or "tracking device" refer to any sensor or marker used to enable spatial tracking and alignment between system components, including but not limited to optical markers, electromagnetic sensors, infrared sensors, acoustic sensors, inertial measurement units, time-of-flight cameras, and other tracking devices that can observe position, orientation, or motion of system components. Registration devices may be mounted on robotic arms, treatment heads, diagnostic probes, patients, or other elements requiring spatial tracking and registration. Registration devices are not required for all system configurations; alternative registration methods described herein can achieve registration without additional hardware.

As used herein, the term "tracking system" refers to something used to observe one or more objects undergoing motion and supply a timely ordered sequence of tracking data (e.g., location data, orientation data, or the like) in a tracking coordinate system for further processing. The tracking system can include electromagnetic tracking that can observe an interventional device equipped with a tracker, optical tracking that can detect reflective markers or light sources, fiber optic shape sensing for flexible catheters and instruments within the body, electro-anatomical mapping, impedance-based tracking, acoustic tracking, lidar, or other tracking modalities that can observe a robotic or manual arm system as it moves or is positioned, or an interventional device as it enters, moves through, and/or exits a patient's body, as well as while outside of the patient's body. The tracking system can employ a single tracking modality or multiple tracking modalities, and multi-modal tracking systems can provide improved accuracy, reliability, and compensation for limitations inherent in individual modalities.

As used herein, the term "tracking data" refers to information recorded by the tracking system related to an observation of one or more objects undergoing motion.

As used herein, the term “object” as used herein can include….

As used herein, the term "head-mounted device," "headset," or "HMD" refers to a display device configured to be worn on the head that has one or more display optics (including lenses) in front of one or more eyes. These terms may be referred to more generally by the term "augmented reality system" or "mixed reality system," where mixed reality refers to interactive environments that combine physical objects with their related data in a manner similar to Medical Internet of Things (MIOT) applications. It should be appreciated that the term "augmented reality system" is not limited to display devices configured to be worn on the head. In some instances, the head-mounted device can also include a non-transitory memory and a processing unit. Examples of suitable head-mounted devices include Microsoft HoloLens® (Microsoft, Redmond, Washington), Magic Leap 2 (Magic Leap, Inc.), DigiLens (DigiLens Inc.) devices, and XREAL Aura (XREAL, Inc.).

As used herein, the extended reality system can enable visualization through various modalities including but not limited to: augmented reality (AR) where virtual content is overlaid on the physical world; virtual reality (VR) where camera passthrough enables viewing of the physical environment within an immersive virtual space; and mixed reality (MR) where physical and virtual elements are combined in an interactive environment. The system can support multiple display types including head-mounted displays, handheld devices such as tablets or smartphones, projector-based systems, and fixed position see-through displays while maintaining registration between virtual content and physical objects.

As used herein, the terms "imaging system," "image acquisition apparatus," or "image acquisition system" refer to technology that creates a visual representation of the interior of a patient's body. For example, the imaging system can be a computed tomography (CT) system, a fluoroscopy system, positron emission computed tomography, magnetic resonance imaging (MRI) system, an ultrasound (US) system including contrast agents, color flow doppler, 4D ultrasound with volume rendering capabilities, or the like.

As used herein, references to specific imaging modalities such as "CT" or "MRI" are intended to be exemplary only. Any reference to a particular imaging modality should be understood to encompass CT, MRI, PET, and other tomographic imaging modalities that can be used interchangeably within the system for visualization, registration, and guidance purposes, unless explicitly stated otherwise.

As used herein, the terms "coordinate system," "augmented reality system coordinate system," or "augmented reality system coordinates" refer to a 3D Cartesian coordinate system that uses one or more numbers to determine the position of points or other geometric elements unique to the particular augmented reality system or image acquisition system to which it pertains. This includes coordinate data from both internal sensors within the headset (such as inertial measurement units and optical sensors) as well as external sensors that may be attached to the headset and/or user for enhanced tracking capabilities. For example, 3D points in the headset coordinate system can be translated, rotated, scaled, or the like, from a standard 3D Cartesian coordinate system.

As used herein, the terms "image data," "imaging dataset," or "imaging data" refer to information recorded by the imaging system related to an observation of the interior of the patient's body. For example, the "image data" or "imaging dataset" can include processed two-dimensional or three-dimensional images or models such as imaging images, e.g., represented by data formatted according to the Digital Imaging and Communications in Medicine (DICOM) standard or other relevant imaging standards.

As used herein, the terms "imaging coordinate system" or "image acquisition system coordinate system" refer to a coordinate system that uses one or more numbers to determine the position of points or other geometric elements unique to the particular imaging system. This includes three-dimensional orthogonal coordinate systems such as Cartesian coordinates, cylindrical coordinates, spherical coordinates, bipolar cylindrical coordinates, bispherical coordinates, confocal ellipsoidal coordinates, and other orthogonal curvilinear coordinate systems, as well as quaternions for representing three-dimensional rotations. For example, points and vectors in the imaging coordinate system can be translated, rotated, scaled, or the like, to the augmented reality system (head mounted displays) coordinate system.

As used herein, the terms "hologram," "holographic," "holographic projection," or "holographic representation" refer to a computer-generated image stereoscopically projected through the lenses of a headset. Generally, a hologram can be generated synthetically in an augmented reality (AR) environment and is not a physical entity.

As used herein, the term "physical" refers to something real. Something that is physical is not holographic (or not computer-generated).

As used herein, the term "two-dimensional" or "2D" refers to something represented in two physical dimensions.

As used herein, the term "three-dimensional" or "3D" refers to something represented in three physical dimensions. An element that is "4D" (e.g., 3D plus a time and/or motion dimension) would be encompassed by the definition of three-dimensional or 3D.

As used herein, the term "integrated" refers to two or more things being linked or coordinated. For example, a diagnostic ultrasound probe can be integrated with a treatment head.

As used herein, the terms "real-time," "near-real-time," or "live" refer to the actual time during which a process or event occurs. In other words, a real-time event is done live (within milliseconds so that results are available immediately as feedback). For example, a real-time event can be represented within 100 milliseconds of the event occurring.

As used herein, the term spatial "registration" refers to the process of determining the spatial relationship between two or more coordinate systems such that positions in one coordinate system can be transformed to corresponding positions in another coordinate system. Registration enables virtual content to be displayed in spatially correct locations relative to real-world objects and may be rigid (preserving distances and angles) or non-rigid (allowing for deformation). In the context of the present disclosure, registration includes steps of transforming tracking and imaging data associated with virtual representations of tracked devices or trajectories — including holographic guides, applicators, and ultrasound image streams — and additional body image data for mutual alignment and correspondence in the head-mounted display coordinate system, enabling a stereoscopic holographic projection display of images and information relative to the body of a physical patient during a procedure.

As used herein, the term "patient coordinate space" refers to a three-dimensional reference frame anchored to the patient's physical anatomy. The patient coordinate space is established through registration of one or more imaging datasets or tracking references to the patient's physical body, such that positions in any other coordinate system including the imaging coordinate system and the augmented reality coordinate system can be expressed as corresponding positions relative to the patient's anatomy. The patient coordinate space accounts for the physical location and orientation of the patient as they exist during the procedure, enabling holographic content, stamped positions, planned therapeutic volumes, and treatment delivery parameters to be maintained in a spatially consistent reference frame that moves with the patient as needed to compensate for patient motion or repositioning.

As used herein, the term "non-contacting energy ablation" refers to procedures that use focused energy delivered from outside the patient body to precisely target and destroy tissue within or on the surface of the patient without invasive surgery. Examples include histotripsy, high-intensity focused ultrasound (HIFU), and other forms of radiotherapy that deliver therapeutic energy without direct contact with the target tissue. It should be appreciated that the systems and methods disclosed herein are not limited to non-contacting procedures and are equally applicable to contact-based percutaneous procedures including but not limited to radiofrequency ablation, microwave ablation, cryoablation, pulsed electric field ablation, and drug delivery systems.

As used herein, the term "sensor fusion" refers to the process of combining data from multiple sensors or sensor modalities to produce a more accurate, reliable, or complete result than could be achieved using any single sensor or modality alone. In the context of tracking, sensor fusion typically combines data from multiple tracking modalities (e.g., electromagnetic, optical, and inertial) to produce position and orientation estimates that compensate for the limitations of individual modalities. Sensor fusion algorithms, such as Kalman filters, can optimally combine complementary information from multiple sensors.

As used herein, the term "inertial measurement unit" or "IMU" refers to a device that measures motion using accelerometers and gyroscopes. A basic IMU can include a three-axis accelerometer and a three-axis gyroscope; some IMUs can also include a three-axis magnetometer, providing orientation information relative to magnetic north. IMUs provide high-frequency motion information with low latency and are immune to metallic interference and line-of-sight occlusion. However, IMUs are subject to drift — small errors in acceleration and angular velocity measurements accumulate over time when integrated to determine position and orientation and accordingly are used in the present disclosure as motion-detection and fail-safe components rather than as standalone absolute position tracking devices. Specifically, IMUs herein are configured to detect changes in position and rotation from a previously established position to trigger re-registration or disable treatment delivery when movement is detected, and not to independently maintain absolute spatial position over time.

As used herein, the term "kinematic solving" refers to the use of known robotic linkage geometry and reported joint or arm position settings to computationally determine the position and orientation of a distal component, such as a treatment head, relative to a proximal base unit, without requiring a separate tracking device mounted on the distal component. Kinematic solving can be employed when the treatment delivery system has its own coordinate system and provides positional and rotational information through an interface or API.

As used herein, the term "planned therapeutic volume" or "PTV" refers to a target tissue contour plus margins defining the region of tissue to receive therapeutic energy delivery. PTVs can be stamped and saved in the system pre-operatively, intra-operatively, or post-operatively, and can be used to assess therapy efficacy, evaluate margin coverage, and prevent over- or under-treatment of target tissues.

As used herein, the term "digital twin" refers to a holographic representation of a physical system component, such as a treatment head or energy delivery system — rendered within the augmented reality environment and constrained to the degrees of freedom and positions permitted by the physical robotic linkage. A digital twin enables a user to plan placement of the physical component by manipulating its holographic counterpart before committing the physical system to a position.

As used herein, the term "bisector configuration" refers to an arrangement of two or more diagnostic ultrasound probes in which the probes are not required to maintain a fixed angular relationship to one another. Unlike biplane imaging systems with fixed probe angles, a bisector configuration allows probes to be positioned at flexible, user-determined angles to provide complementary imaging perspectives, with one probe optionally dedicated to treatment delivery monitoring and another to targeting or surveillance, simultaneously or in an interleaved fashion.

As used herein, the term "segmentation" refers to the process of identifying and extracting specific structures from medical images. Segmentation may be manual (user-delineated), semi-automatic (user-guided with automatic processing), or fully automatic (algorithm-based without user intervention). Segmentation produces a three-dimensional representation of anatomical structures such as organs, bones, vessels, or pathological structures such as tumors. Segmented structures can be visualized as holographic overlays in the augmented reality environment.

As used herein, the term "multi-planar reconstruction" or "MPR" refers to a technique for visualizing medical imaging data as two-dimensional slices in arbitrary orientations. While medical images are typically acquired in standard planes (axial, sagittal, coronal), multi-planar reconstruction allows visualization of slices in any orientation, including oblique planes aligned with anatomical structures or interventional devices.

It has been surprisingly found that the systems and methods of the present invention provide continuous spatial alignment between a holographic augmented reality visualization system and a therapeutic delivery system throughout all procedural phases (e.g, localization, planning, and treatment) without requiring repeated manual registration or separate intermediate tracking components at each phase transition. This continuous registration architecture resolves a previously unmet need in non-contacting and percutaneous procedure guidance, where the disjointed transition between procedural phases, particularly following repositioning of a robotic arm or application of a coupling membrane, has historically required the physician to mentally re-correlate the treatment zone with the patient anatomy without spatial continuity.

The present technology provides a system for guiding a medical procedure comprising an augmented reality visualization system configured to display a holographic representation of the procedure environment through a head-mounted device. The system further comprises a treatment delivery system including a robotic linkage and a therapeutic delivery tool. The therapeutic delivery tool may be selected from the group consisting of non-contacting energy delivery devices, percutaneous devices, ablation devices, and drug delivery devices, enabling the system to be applied across a broad range of therapeutic modalities without modification to the core registration and visualization architecture. A registration tracking system including one or more tracking sensors is configured to establish a common coordinate system shared by the augmented reality visualization system and the treatment delivery system. The registration tracking system maintains continuous spatial alignment between the augmented reality visualization system and the treatment delivery system throughout the localization, planning, and treatment phases of the medical procedure. The augmented reality visualization system is configured to display a holographic representation of the therapeutic energy beam of the therapeutic delivery tool relative to the tracked position and orientation of the therapeutic delivery tool as viewed through the head-mounted device, providing the physician with direct spatial context of the energy delivery pathway relative to the patient anatomy at all times during the procedure.

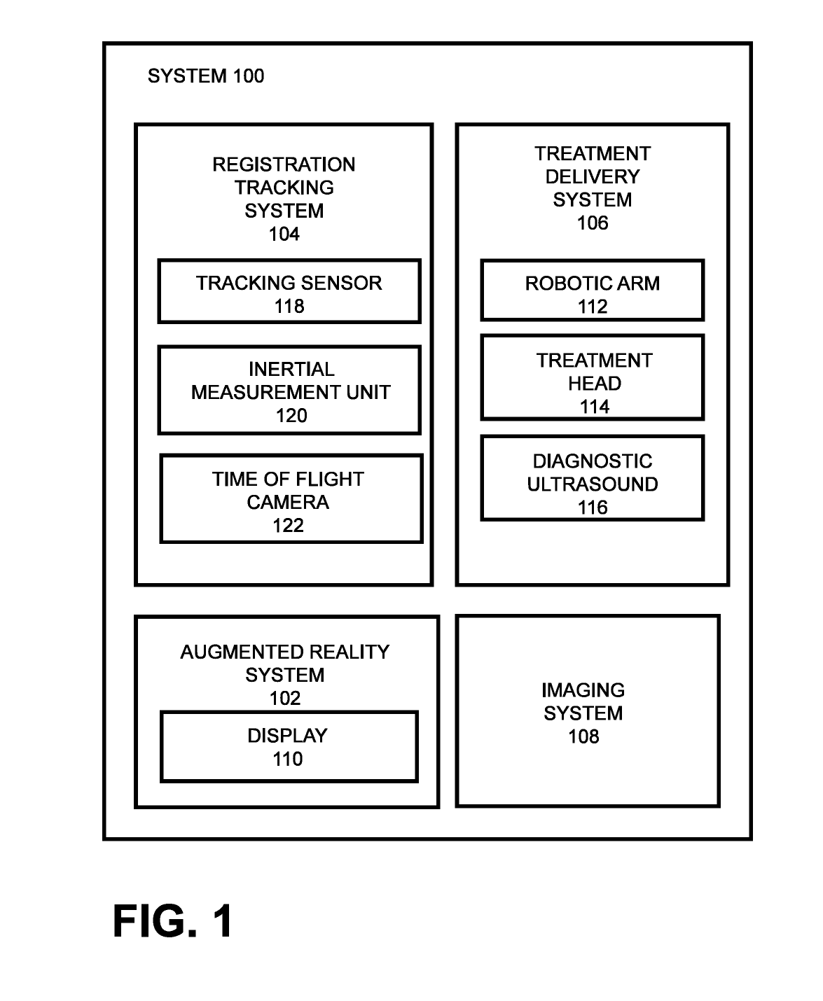

With reference to FIG. 1, a system for non-contacting energy ablation and percutaneous procedure guidance 100 is shown. The system 100 can include an augmented reality visualization system 102, a registration tracking system 104, a treatment delivery system 106, and an imaging system 108. These four subsystems can work together to enable visualization, tracking, delivery, and monitoring of therapeutic procedures through continuous spatial registration and data integration.

The augmented reality visualization system 102 can be configured to display holographic representations of relevant procedural and anatomical information. The registration tracking system 104 can maintain continuous spatial alignment between the augmented reality visualization system 102, the treatment delivery system 106, and optionally, the imaging system 108 through various tracking modalities and registration devices. The treatment delivery system 106 can deliver therapeutic energy or place therapeutic devices at target tissue locations. The imaging system 108 can provide both real-time intraoperative visualization and pre-operative imaging data for use throughout the procedure.

The system 100 can be applied across three procedural phases. The localization phase can involve using diagnostic ultrasound to locate and identify target tissue under image guidance. The planning phase can include both pre-operative and intraoperative determination of treatment zones and approach development, while considering equipment positioning and coupling mechanisms, with capability for retrospective assessment through stored procedural data. The treatment phase can involve delivery of therapeutic energy or device placement while monitoring the treatment zone and managing factors such as respiratory motion, table movement, patient movement, and internal organ or anatomical structure movement or deformation.

Although the present disclosure describes the system 100 in the context of non-contacting energy ablation procedures, the registration tracking system, augmented reality visualization capabilities, and treatment delivery mechanisms described herein can be applied to any capital equipment system involving a robotic or manual linkage between a base unit and a therapeutic delivery tool. Non-limiting examples of applicable systems can include histotripsy systems, high-intensity focused ultrasound (HIFU) systems, radiotherapy systems such as CyberKnife and GammaKnife, radiofrequency ablation systems, microwave ablation systems, cryoablation systems, pulsed electric field ablation systems, and drug delivery systems. The system 100 can be applied to both non-contacting and contact-based percutaneous procedures. Percutaneous devices can include, for example, percutaneous needles, biopsy needles, drainage catheters, and trocar-based access systems. Ablation devices can include, for example, radiofrequency ablation probes, microwave ablation antennas, cryoablation probes, pulsed electric field ablation catheters, laser ablation devices, and high-intensity focused ultrasound (HIFU) and histotripsy systems. Drug delivery devices can include, for example, drug-eluting needles, intratumoral injection systems, catheter-based infusion systems, and robotic-arm-mounted injection devices configured to deliver chemotherapeutic, immunotherapeutic, or other pharmacological agents to a target tissue volume under image guidance.

The registration tracking system 104 can include data storage and management components configured to record, analyze, and retrieve procedural data throughout all phases of the procedure. Recorded data can include pre-operative planning data, intraoperative treatment data, real-time ultrasound streams, robotic arm positioning data, treatment delivery parameters, respiratory motion tracking data, and simulation results. This data management capability can enable comprehensive procedure documentation, outcome analysis, and quality assessment, and can support both real-time procedure guidance and post-procedure review.

The augmented reality visualization system 102 can include a head-mounted device 110. The head-mounted device 110 can function both as an output device, presenting virtual content overlaid on or integrated with a view of the real-world environment, and as a sensing and tracking device, using built-in sensors to determine its own position and orientation within the registration tracking system 104 coordinate space. These two functions can operate simultaneously and interdependently, as the accuracy of holographic visualization can depend directly on the sensing accuracy of the head-mounted device 110 itself.

Built-in sensing capabilities of the head-mounted device 110 can include cameras for visual environment tracking through simultaneous localization and mapping (SLAM), inertial measurement units for head motion tracking, depth sensors for three-dimensional environment geometry capture, and other sensors enabling the device to maintain spatial awareness within the procedure environment. The head-mounted device 110 can use these built-in sensors to track its position and orientation within the room, enabling display of holographic content at fixed locations in room coordinates that appear stationary as the user moves. The sensing capabilities of the head-mounted device 110 can additionally be leveraged for optical tracking of registration devices, surface scanning for anatomical registration, and detection of patient motion, reducing or eliminating the need for separate external sensing hardware for certain registration functions.

The head-mounted device 110 can be implemented as a head-mounted display (HMD) worn by a physician or medical professional during a procedure, a handheld display device such as a tablet or smartphone, a projector-based system projecting virtual content onto physical surfaces, or other display technologies capable of augmented reality visualization. In some embodiments, the augmented reality visualization system 102 can include multiple head-mounted devices 110 operating simultaneously, enabling different members of the procedural team to receive visualization appropriate to their respective roles.

The head-mounted device 110 can use optical see-through technology, where virtual content is displayed on transparent or semi-transparent optics through which the user directly views the real world, or video see-through technology, where cameras capture real-world video displayed to the user with virtual content composited into the video stream. Optical see-through can provide little to no latency in the real-world view and no dependence on camera quality. Video see-through can provide precise alignment between real-world video and virtual content and can enable image processing of the real-world view. Suitable head-mounted devices 110 can include the Microsoft HoloLens (first and second generation), Magic Leap One and Magic Leap 2, Meta Quest Pro, Varjo XR-3, and other augmented reality or mixed reality headsets. Selection factors can include field of view, display resolution, display brightness, tracking accuracy, processing power, comfort, battery life, and cost.

The augmented reality visualization system 102 can support multiple extended reality modalities through the head-mounted device 110. In augmented reality mode, virtual content can be overlaid on the physician's direct view of the physical world through transparent or semi-transparent optics. In mixed reality mode, physical and virtual elements can be combined in an interactive environment enabling the user to interact with both physical and holographic objects simultaneously. In virtual reality mode, camera passthrough can enable viewing of the physical environment within an immersive virtual space, which can be useful for procedure planning and simulation workflows. The system 100 can support switching between these modalities based on procedural phase or user preference.

The head-mounted device 110 can include processing hardware for executing application software. Processing hardware can include central processing units (CPUs), graphics processing units (GPUs), digital signal processors (DSPs), application-specific integrated circuits (ASICs), field-programmable gate arrays (FPGAs), or other processing devices. Processing can occur on processors within the head-mounted device 110 itself, on external computing devices such as desktop computers, laptop computers, mobile devices, or servers, or distributed across multiple devices communicating via a network.

The augmented reality visualization system 102 can display various types of holographic content through the head-mounted device 110. Holographic content can include two-dimensional and three-dimensional representations of live diagnostic ultrasound beams projected in the patient coordinate space as a holographic flashlight, pre-planned treatment zones and planned therapeutic volumes, segmented anatomical structures rendered as three-dimensional surface meshes, multi-planar reconstruction (MPR) slices positioned in three-dimensional space at their corresponding anatomical locations, critical structure warnings and overlap alerts, real-time treatment delivery feedback, virtual representations of tracked instruments and robotic arm positions, finite element analysis (FEA) and computational fluid dynamics (CFD) simulation results, and role-specific guidance information. Visual appearance of holographic content can be controlled through color, transparency, brightness, and rendering style including solid, wireframe, and point cloud representations.

The augmented reality visualization system 102 can support multiple user interaction modalities through the head-mounted device 110. Hand gesture interaction can enable the user to manipulate holographic content, adjust visualization parameters, and confirm procedural actions without physical controllers. Voice command interaction can enable the user to issue spoken commands to control system functions while maintaining sterile field integrity. Controller-based interaction can provide an alternative input method for situations where gesture or voice interaction is not optimal. The head-mounted device 110 can support gaze-based interaction, where the direction of the physician's gaze can be used to select holographic content or trigger system actions, including as a safety mechanism to disable treatment delivery when the physician is not actively monitoring the treatment zone.

The augmented reality visualization system 102 can support role-specific visualization, where different members of the procedural team receive content through their respective head-mounted devices 110 tailored to their individual responsibilities. A table-side assistant can receive a visualization oriented toward monitoring treatment zone positioning and physical system components. A console operator can receive a visualization oriented toward system control, robotic arm positioning, and treatment parameter management. Additional team members such as anesthesia or nursing staff can receive simplified status displays providing relevant procedural information without full guidance overlays. Each role-based display can be configured independently with respect to content type, visualization parameters, and interaction modalities.

The augmented reality visualization system 102 can support remote visualization configurations, where a physician or specialist who is not physically present in the procedural environment can receive procedural information through a networked connection to a remote head-mounted device 110 or other display. In remote configurations, the remote user can view holographic representations of the treatment zone, imaging data, robotic arm positioning, and real-time treatment status. The remote user can provide guidance, approve treatment parameters, or perform a virtual sign-off on treatment initiation through the remote display interface. Remote visualization can enable procedural support in outpatient, mobile, or distributed clinical settings where on-site specialist presence is not available or required.

The treatment delivery system 106 can deliver therapeutic energy or place therapeutic devices at target tissue locations under image guidance. The treatment delivery system 106 can include a robotic arm 112 with an integrated treatment head 114 and an integrated diagnostic ultrasound probe 116. In some embodiments, the robotic arm 112 can be replaced by a manual arm or other mechanical linkage, and the systems and methods described herein can apply equally to manual arm configurations. The treatment delivery system 106 can be a standalone capital equipment system or can be integrated with existing hospital imaging infrastructure.

The treatment delivery system 106 can support therapeutic modalities including histotripsy, high-intensity focused ultrasound (HIFU), radiotherapy systems such as robotic radiosurgery platforms, radiofrequency ablation, microwave ablation, cryoablation, pulsed electric field ablation, and drug delivery systems. The systems and methods described herein can be applicable to any capital equipment system involving a robotic or manual linkage between a base unit and a therapeutic delivery tool, whether contacting or non-contacting in nature. The treatment delivery system 106 operates within a common coordinate system established and maintained by the registration tracking system 104, described in detail herein. This spatial registration between the treatment delivery system 106 and the head-mounted device 110 enables the holographic visualization, position stamping, and automated delivery capabilities.

The robotic arm 112 can provide motorized multi-axis positioning of the treatment head 114 relative to the patient and can be configured to move through pre-defined treatment patterns during automated therapy delivery. The treatment head 114 can include the terminal component of the robotic arm 112 configured to emit or deliver therapeutic energy to a target tissue volume, such as a focused ultrasound transducer array, ablation probe, or radiation delivery element. The robotic arm 112 can include a base unit and one or more articulating linkages connecting the base unit to the treatment head 114. The base unit can maintain a fixed position relative to the procedure table during treatment delivery, providing a stable mechanical reference from which the position and orientation of the treatment head 114 can be determined. In some embodiments, the treatment delivery system 106 can provide positional and rotational data for each articulating linkage of the robotic arm 112 through an interface or application programming interface (API), enabling kinematic solving of the treatment head 114 position.

The focused ultrasound transducer 116 can comprise an array of piezoelectric elements configured to emit and focus acoustic energy at a defined focal point or focal zone within target tissue. The focused ultrasound transducer can operate in both diagnostic and therapeutic modes: in diagnostic mode, the transducer can generate real-time ultrasound imaging of target anatomy to facilitate localization and treatment planning; in therapeutic mode, the transducer can deliver focused acoustic energy to a target tissue volume to induce thermal ablation, mechanical disruption, or other therapeutic effects without requiring percutaneous access. The focal point depth, beam geometry, and energy delivery parameters of the focused ultrasound transducer can be controlled by the treatment delivery system 106, and the position and orientation of the transducer can be tracked by the tracking sensors 118 to enable registration of the ultrasound beam with the augmented reality visualization system 102 and display of the corresponding holographic flashlight through the head-mounted device 110.

The robotic arm 112 can support freedrive operation, where the arm can be manually moved by the physician or assistant to a desired position before locking into place for treatment delivery. Physical controls for freedrive operation can include a space mouse or equivalent input device. The robotic arm 112 can also support automated positioning, where the system 100 directs the arm to move to a pre-planned or algorithm-suggested position without manual input. Safety interlocks can prevent robotic arm 112 motion beyond predefined workspace boundaries and can halt motion when unexpected resistance is detected.

The treatment head 114 can be detachably coupled to the robotic arm 112 and can serve as the primary energy delivery component of the treatment delivery system 106. The treatment head 114 can include one or more transducers configured to focus therapeutic ultrasound energy at a defined focal point within the target tissue. The focal point depth, size, and energy parameters can be adjustable based on target tissue type, depth, and treatment requirements. The treatment head 114 can include a coupling membrane that provides acoustic coupling between the patient and the treatment head 114. The coupling membrane can include a flexible membrane that conforms to the patient's anatomy to allow uninterrupted ultrasound delivery. In histotripsy and HIFU applications, the coupling membrane can contain an ultrasound-transmitting medium and can be designed for straightforward setup and replacement. Application of the coupling membrane requires the robotic arm 112 to be temporarily moved away from the target position and subsequently returned.

The treatment head 114 can include physical control interfaces for managing treatment delivery parameters. Physical controls can include planning knobs for setting planning parameters in the x, y, and z axes, voltage adjustment controls for setting energy delivery parameters, a trackpad for cursor control and navigation, and an emergency stop button for immediate cessation of treatment delivery. A software interface can provide procedural phase guidance, current task instructions, image plane customization, DICOM view controls, and axis perspective controls for use in conjunction with the physical controls.

The diagnostic ultrasound probe 116 can be integrated with the treatment head 114 and can provide real-time ultrasound imaging of target tissue throughout the localization, planning, and treatment phases. The diagnostic ultrasound probe 116 can be a curvilinear, linear, or phased array probe selected based on the procedural application and target tissue depth. Integration of the diagnostic ultrasound probe 116 with the treatment head 114 can enable simultaneous or interleaved acquisition of therapeutic energy delivery and real-time imaging feedback without requiring repositioning of either component between imaging and treatment activities.

In some embodiments, the treatment delivery system 106 can include a bisector configuration with two or more diagnostic ultrasound probes 116 positioned at flexible, user-determined angles relative to the treatment head 114. Unlike fixed biplane imaging configurations, the bisector configuration can allow probes to be repositioned to provide complementary imaging perspectives based on target location and acoustic window availability. In a bisector configuration, one probe can be dedicated to treatment delivery monitoring while another provides targeting or surveillance imaging, operating simultaneously or in an interleaved fashion. The bisector configuration can support volume reconstruction from live tracked two-dimensional ultrasound sweeps acquired by either probe, providing three-dimensional imaging data without requiring a dedicated four-dimensional ultrasound system.

Once the head-mounted device 110 and the treatment delivery system 106 are registered within a common coordinate system through the registration tracking system 104, the system 100 can save and stamp the position and orientation of the treatment head 114 or robotic arm 112 in six degrees of freedom. Position stamping can enable the treatment head 114 to return to a previously established position and orientation after the coupling membrane is applied or after any portion of the system is moved, maintaining precise alignment with the previously localized target. The system 100 can also stamp the position and orientation of a freehand diagnostic probe during the localization phase, enabling the treatment head 114 to subsequently be guided to that position by the robotic arm 112.

The system 100 can display, through the head-mounted device 110, a holographic light ray transecting or emanating from the treatment head 114 or probe head and projected in the patient coordinate space to represent the planned trajectory and facilitate alignment. The system 100 can stamp and save planned therapeutic volumes (PTVs), defined as target contours plus margins, using the ultrasound flashlight from either the freehand probe or the integrated diagnostic ultrasound probe 116 to hold regions of interest in three-dimensional space. PTV stamping can be performed pre-operatively, intra-operatively, or post-operatively to help determine therapy efficacy, assess margin coverage, and prevent over- or under-treatment of target tissues. Stored PTVs can be linked to or compared with treatment monitoring data to analyze therapy delivery outcomes in real time or post-treatment.

The system 100 can enable a digital twin of the treatment head 114 and energy delivery system to be displayed through the head-mounted device 110 prior to physical positioning. The user can manipulate the holographic digital twin to plan the desired therapeutic position and configuration, with manipulation constrained to the degrees of freedom and positions permitted by the physical robotic linkage of the robotic arm 112. Once a desired configuration is determined through digital twin manipulation, the physical treatment head 114 can be moved into the planned position. This capability can be particularly valuable in cases with limited treatment head placement options or restricted space around the patient.

The system 100 can incorporate a probe placement algorithm configured to suggest optimal placement locations for the treatment head 114 and diagnostic ultrasound probe 116. The probe placement algorithm can accept as primary inputs the image-based data from the imaging system 108 and the target location within that imaging data. The algorithm can additionally accept optional inputs including tissue type along the acoustic pathway, coupling kit fluid level, nearby protected structure locations, and probe type specifications to refine suggested placements. The probe placement algorithm can identify placement locations that provide a clear acoustic path to the target for energy delivery while minimizing potential interaction with nearby protected structures. The algorithm can generate multiple saved placement locations, enabling the physician to switch between positions optimized for different aspects of the procedure such as diagnostic imaging quality and therapeutic energy delivery efficiency. The probe placement algorithm can validate suggested positions against the mechanical constraints of the robotic arm 112 to confirm that suggested placements are physically achievable before the physician commits to a position.

The system 100 can display the acoustic pathway 124 of the therapeutic ultrasound beam from the treatment head 114 to the target tissue as a holographic overlay through the head-mounted device 110. The acoustic pathway visualization can display either a partial or complete representation of the ultrasound beam path, enabling clinicians to evaluate how the therapeutic energy will interact with different tissue structures along the beam path prior to treatment delivery. Similar to radiation beam therapy planning, the system 100 can indicate where additional energy compensation may be needed when the therapeutic beam encounters bones or other acoustically dense structures in the pathway. This visualization can assist clinicians in planning optimal acoustic windows for energy delivery, understanding the relationship between tissue composition and required energy parameters, and identifying paths that minimize interaction with high-density structures while maintaining effective access to the target tissue.

During the treatment phase, the treatment delivery system 106 can provide fully automated dynamic movement of the treatment head 114 through optimized patterns covering the planned treatment volumes. The robotic arm 112 can continuously move the treatment head 114 through the pre-defined treatment pattern while the system 100 provides real-time monitoring of imaging and treatment status. Automated treatment delivery can be initiated by the physician after confirmation of system registration, target localization, and treatment zone planning, and can proceed without requiring continuous manual input during energy delivery.

Safety interlocks can be integrated into the automated treatment process. The system 100 can disable treatment delivery when unexpected motion of the robotic arm 112 is detected, when the physician's gaze is detected to be directed away from the treatment zone, or when the planned treatment delivery zone is determined to overlap with a marked protected structure. Treatment delivery can resume only after the physician confirms re-registration of the system or confirms that treatment zone alignment is maintained.

The registration tracking system 104 can establish and maintain a common coordinate system between the head-mounted device 110, the treatment delivery system 106, and the imaging system 108. By establishing this common coordinate system, the registration tracking system 104 can enable holographic content displayed through the head-mounted device 110 to appear spatially aligned with the robotic arm 112, the treatment head 114, the diagnostic ultrasound probe 116, and patient anatomy throughout all procedural phases. Registration can be established initially before a procedure begins and can be updated continuously or on a triggered basis throughout the procedure to account for patient motion, respiratory motion, table movement, or other factors that may affect spatial alignment.

The registration tracking system 104 can achieve registration without requiring a separate intermediate tracking component in all configurations. In some embodiments, the built-in sensing capabilities of the head-mounted device 110, including its internal cameras, depth sensors, and inertial measurement units, can be used directly to establish and maintain registration between the head-mounted device 110 and the treatment delivery system 106. In other embodiments, external tracking sensors 118 or tracking systems can supplement or replace the built-in sensing capabilities of the head-mounted device 110. The system 100 can support hybrid approaches combining multiple tracking modalities to leverage complementary strengths and compensate for limitations inherent in any single modality.

The registration tracking system 104 can employ one or more tracking sensors 118 positioned at various locations within the system 100 and the procedure environment to establish and maintain the common coordinate system. Tracking sensors 118 can be mounted on the head-mounted device 110 itself, on the robotic arm 112 or treatment head 114, on the diagnostic ultrasound probe 116, on the patient, or at fixed positions within the operating space. The system 100 can employ multiple tracking sensors 118 simultaneously across multiple tracking modalities, including optical, electromagnetic, infrared, acoustic, inertial, and time-of-flight sensing modalities, with each tracking sensor 118 contributing position and orientation data that is combined through sensor fusion to produce a unified spatial registration across all system components.

The registration tracking system 104 can employ optical tracking sensors 118 to establish and maintain registration between the head-mounted device 110 and the treatment delivery system 106. Optical tracking sensors 118 can use cameras or other optical sensors to determine the position and orientation of tracked objects based on visual information. Optical tracking sensors 118 can be active, using markers that emit infrared light, or passive, using retroreflective markers that reflect light from an illuminator. Optical tracking sensors 118 can also use image target detection, where printed two-dimensional patterns are recognized by computer vision algorithms, or model target detection, where three-dimensional models of physical objects are matched against observed camera images.

In one embodiment, an external optical tracking sensor 118 can be positioned within the operating space to track both the head-mounted device 110 and the treatment head 114 or robotic arm 112 simultaneously, establishing a common coordinate system between them through a shared external reference frame. In another embodiment, an optical tracking sensor 118 integrated into the head-mounted device 110 can track optical markers mounted on the treatment head 114 or robotic arm 112 directly, enabling the head-mounted device 110 to determine the position and orientation of the treatment delivery system 106 relative to itself without requiring separate external tracking infrastructure. An optical tracking sensor 118 mounted on the head-mounted device 110 can also track an electromagnetic field generator positioned on or near the procedure table, establishing registration between the electromagnetic tracking coordinate system and the head-mounted device 110 coordinate system. Optical tracking sensors 118 can have a line-of-sight requirement, and the system 100 can incorporate alternative or supplementary tracking sensors 118 operating under other modalities to maintain registration when line-of-sight is interrupted.

The registration tracking system 104 can employ electromagnetic tracking sensors 118 configured to determine position and orientation by measuring electromagnetic fields generated by a field generator. Electromagnetic tracking sensors 118 can offer the advantage of not requiring line-of-sight between the tracking sensor 118 and the field generator, allowing the tracking sensor 118 to be tracked even when occluded by the patient's body, surgical drapes, or other obstructions. The system 100 can account for electromagnetic field distortion caused by metallic objects in the procedure environment through sensor fusion or field distortion compensation algorithms.

In some embodiments, a tracking sensor 118 can be an electromagnetic tracking sensor 118 which can be removable and reusable and can be configured for attachment to various components of the system 100 or to the patient. The electromagnetic tracking sensor 118 can include a sterilizable receptacle, a snap-fit attachment mechanism, a backstop, and a keyed groove ensuring repeatable and consistent positioning across uses. The electromagnetic tracking sensor 118 can be cabled or wireless, including battery-powered wireless configurations. In addition to electromagnetic sensing elements, the tracking sensor 118 can incorporate other sensor types including inertial measurement units, acoustic sensors, optical codes, and active or passive infrared sensors, with suitable calibration for each sensor type included.

Electromagnetic tracking sensors 118 can be mounted on the robotic arm 112, the treatment head 114, the diagnostic ultrasound probe 116, the patient, or other components requiring spatial tracking. When mounted on the diagnostic ultrasound probe 116, the tracking sensor 118 can be attached using a bracket configured for compatibility with curvilinear, linear, and phased array probe types. The bracket can maintain a fixed and known spatial relationship between the tracking sensor 118 and the ultrasound image plane, established through calibration, with each device having a unique three-dimensional coordinate offset determined by its device identifier and calibration data. An electromagnetic field generator can be positioned under or beside the patient to establish the electromagnetic tracking volume within the procedure environment.

The registration tracking system 104 can support additional tracking sensors 118 operating under modalities beyond optical and electromagnetic tracking. Infrared tracking sensors 118 can use one or more active infrared light-emitting diodes or passive retroreflective infrared markers to determine position and orientation. An infrared tracking sensor 118 can be mounted on the head-mounted device 110 to directly track the treatment head 114 or robotic arm 112, providing registration between the head-mounted device 110 and the treatment delivery system 106 through a direct sensing relationship without requiring separate external tracking infrastructure. An infrared tracking sensor 118 can alternatively be mounted within the operating space in a fixed position, tracking markers on the head-mounted device 110 and the treatment delivery system 106 simultaneously.

One or more acoustic tracking sensors 118 can use ultrasonic sound waves to determine position through time-of-flight measurements and triangulation, and can be mounted on the head-mounted device 110, the robotic arm 112, or at fixed positions in the operating space. Radio-frequency based tracking sensors 118 can include ultra-wideband (UWB), Bluetooth Low Energy (BLE), and other modalities providing positioning information through time-of-flight or signal strength measurements. Fiber optic shape sensing can use fiber optic cables with embedded sensors to reconstruct the three-dimensional shape of flexible instruments, which can be applicable to catheter-based or flexible device embodiments of the treatment delivery system 106. The system 100 can support any combination of tracking sensors 118 operating simultaneously across these modalities, with sensor fusion algorithms combining data from multiple tracking sensors 118 to produce position and orientation estimates with improved accuracy and reliability relative to any single modality.

The registration tracking system 104 can include one or more inertial measurement units 120 mounted on the robotic arm 112, the treatment head 114, or other components of the treatment delivery system 106. The inertial measurement unit 120 can function as a tracking sensor 118 under the inertial sensing modality and can include a three-axis accelerometer and a three-axis gyroscope. In some embodiments, the inertial measurement unit 120 can also include a three-axis magnetometer providing orientation information relative to magnetic north.

In certain embodiments of the present technology, the inertial measurement unit 120 can function as a motion-detection and fail-safe component rather than as a standalone absolute position tracking device. The inertial measurement unit 120 can be configured to detect changes in position and rotation of the robotic arm 112 or treatment head 114 from a previously established and registered position. Upon detecting motion exceeding a defined threshold, the inertial measurement unit 120 can trigger re-registration of the treatment delivery system 106 within the common coordinate system, can alert the physician through the head-mounted device 110, or can disable treatment delivery until registration is reconfirmed. The inertial measurement unit 120 can operate at high update rates, typically 100 Hz to 1000 Hz or higher, providing low-latency motion detection with immunity to metallic interference and line-of-sight occlusion. The inertial measurement unit 120 can operate in combination with other tracking sensors 118 through sensor fusion to compensate for drift inherent in inertial sensing when used as a standalone position tracking method.

In some embodiments, the registration tracking system 104 can determine the position and orientation of the treatment head 114 relative to the base unit of the robotic arm 112 through kinematic solving rather than through a tracking sensor 118 mounted directly on the treatment head 114. Kinematic solving can use the known geometry of the articulating linkages of the robotic arm 112, together with joint position or arm configuration data provided by the treatment delivery system 106 through an interface or application programming interface (API), to compute the position and orientation of the treatment head 114 in the coordinate system of the base unit. When the base unit of the robotic arm 112 is itself registered within the common coordinate system through a tracking sensor 118 mounted on or near the base unit, kinematic solving can extend this registration to the treatment head 114 without requiring a separate tracking sensor 118 mounted on the treatment head 114 itself. Kinematic solving can be combined with inertial measurement unit 120 data as a cross-check, where the inertial measurement unit 120 can detect motion events that may require kinematic recalculation or re-registration.

The registration tracking system 104 can include a time-of-flight camera 122 functioning as a tracking sensor 118 under the depth sensing modality. The time-of-flight camera 122 can be mounted on the treatment head 114 or other components of the treatment delivery system 106, or at a fixed position within the operating space. The time-of-flight camera 122 can emit near-infrared light and measure the time required for reflected light to return to the sensor, generating a depth map representing the three-dimensional geometry of surfaces within its field of view.

In one embodiment, the time-of-flight camera 122 can be directed toward the patient's chest wall or abdomen and can measure the motion of the body surface during respiration. This respiratory motion data can be used to synchronize treatment delivery with the respiratory cycle, can trigger treatment gating at defined respiratory phases, or can provide input to robotic arm 112 motion compensation algorithms that continuously adjust the position of the treatment head 114 to maintain spatial alignment with a moving target during breathing. The time-of-flight camera 122 can also contribute depth sensing data for surface scanning and anatomical registration purposes, supplementing or replacing other tracking sensors 118 in configurations where surface-based registration is preferred.

The registration tracking system 104 can include data storage and management capabilities configured to record and retrieve procedural data throughout all phases of the procedure. Stored data can include pre-operative planning data, coordinate transformations established during registration, intraoperative treatment data, real-time ultrasound image streams with associated position and orientation data from tracking sensors 118, robotic arm 112 positioning data, treatment delivery parameters, respiratory motion tracking data, inertial measurement unit 120 motion logs, and simulation results. Coordinate transformations can be stored alongside procedural data to preserve the spatial relationships between coordinate systems as they existed at each point during the procedure.

Stored data can be retrieved and replayed to reconstruct the procedural guidance view at any point during or after the procedure. Retrospective review can support post-procedure assessment of treatment zone coverage, instrument trajectory accuracy, and quality assurance. The system 100 can record the planned therapeutic volume (PTV) and compare it to the delivered treatment zone during or after the procedure to assess margin coverage and identify areas of potential under-treatment or critical structure proximity. Data storage can reside within the head-mounted device 110, on an external computing device communicating via a network, or on a remote server or cloud-based storage system. Access to stored procedural data can be controlled through authentication mechanisms to maintain patient data privacy and comply with applicable healthcare data security requirements.

The imaging system 108 can provide real-time intraoperative visualization through diagnostic ultrasound, as well as pre-operative imaging data from CT, MRI, and other tomographic modalities, for use throughout the localization, planning, and treatment phases. The imaging system 108 can allow physicians to evaluate both the target tissue and critical anatomy in the area of interest through real-time diagnostic ultrasound alongside pre-operative imaging data. The imaging system 108 can communicate with the registration tracking system 104 and the augmented reality visualization system 102 to enable holographic display of imaging data in the patient coordinate space as established through registration.

The imaging system 108 can incorporate real-time diagnostic ultrasound imaging using the diagnostic ultrasound probe 116 integrated with the treatment head 114, a freehand diagnostic ultrasound probe, or both. The imaging system 108 can support curvilinear, linear, and phased array probe types selected based on the procedural application and target tissue depth. The imaging system 108 can enable multi-planar viewing with rotating and translating diagnostic ultrasound capabilities to support full volumetric targeting of the region of interest.

The imaging system 108 can support simultaneous connection of two or more diagnostic ultrasound probes 116, enabling complementary imaging perspectives to be acquired and displayed either simultaneously or in an interleaved fashion. When two probes are connected, the imaging system 108 can display both ultrasound flashlights simultaneously through the head-mounted device 110, or can alternate between displaying the ultrasound image from one probe followed by the other through interleaved operation. The two connected probes can be of the same or different types, as complementary imaging perspectives can be achieved through differences in probe characteristics, positioning, and acoustic windows as well as through angular separation. In a bisector configuration, one probe can be dedicated to treatment delivery monitoring while the other is used for targeting or surveillance, operating simultaneously or in an interleaved fashion.

The imaging system 108 can incorporate pre-operative medical imaging including CT, MRI, PET, and other tomographic modalities that can be brought into the system and projected to the patient coordinate space. References to specific imaging modalities such as CT or MRI are intended to be exemplary only, and any such reference can be understood to encompass CT, MRI, PET, and other tomographic imaging modalities that can be used interchangeably within the imaging system 108 for visualization, registration, and guidance purposes.

The imaging system 108 can enable fusion of pre-operative medical imaging with real-time diagnostic ultrasound by adjusting multi-planar images to match the stationary real-time ultrasound image, relating the imaging coordinate system to the coordinate system of the tracked ultrasound beam. Since the position of the tracked ultrasound image is known in the head-mounted device 110 coordinate system through registration, a transformation can be determined to map from imaging coordinates to head-mounted device 110 coordinates. This registration can also be performed manually by aligning a segmented skin surface mesh extracted from pre-operative imaging with the actual skin surface of the patient, where the user can translate and rotate the holographic skin mesh until it matches the patient's real skin surface as viewed through the head-mounted device 110. The imaging system 108 can support use of pre-operative imaging in both segmented and unsegmented forms depending on procedural requirements and available preparation time.

Pre-operative imaging in the imaging system 108 can be used in multiple configurations depending on available modalities and procedural complexity. In a comprehensive configuration, CT and MRI imaging can both be acquired, fused through image fusion algorithms, and segmented to produce three-dimensional surface models of target structures and surrounding anatomy. In configurations requiring less preparation time, CT and MRI can be used without fusion or segmentation, with multi-planar reconstruction slices generated independently from each modality and displayed in the head-mounted device 110. In the most streamlined configuration, a single modality such as CT can be used without fusion or segmentation to generate multi-planar reconstruction slices for procedural guidance.

The imaging system 108 can generate three-dimensional volumetric data from live tracked two-dimensional ultrasound sweeps acquired by a freehand or robotically positioned diagnostic ultrasound probe 116. As the probe is moved through a sweep across the region of interest, the imaging system 108 can record each two-dimensional ultrasound image alongside its position and orientation as determined by the tracking sensor 118 mounted on or associated with the probe. The imaging system 108 can reconstruct a three-dimensional volume from these sequentially acquired and spatially registered two-dimensional slices. This volume reconstruction capability can provide three-dimensional ultrasound imaging data without requiring a dedicated four-dimensional ultrasound probe, and can be used in conjunction with the holographic visualization capabilities of the augmented reality visualization system 102 to display the reconstructed volume in the patient coordinate space.

The imaging system 108 can also support volumetric data generated directly from four-dimensional ultrasound probes when available. Additional visualization elements applicable to volumetric ultrasound data can include positional markers, clock face position indicators, distance measurements, trajectory projections, and tissue deformation data.

The imaging system 108 can support integration with automated breast ultrasound systems, including systems incorporating mechanically driven or robotically driven ultrasound probe arrays configured for comprehensive breast coverage. In this embodiment, the diagnostic ultrasound probe 116 can be integrated into a mechanical boom or articulatable arm and can be operated either manually or robotically. The registration tracking system 104 can relate the coordinate systems of the automated breast ultrasound system and the head-mounted device 110 to the anatomy and target through the same registration processes, enabling holographic display of live imaging data, three-dimensional reconstructions, and additional visual elements for subsequent treatment planning.

Automated breast ultrasound system integration can support AI-powered capabilities including scan quality assessment for evaluating breast coverage and probe positioning, and automated anatomical feature detection to enable consistency and accelerate examinations. Specialized probe designs configured for breast imaging, such as probes with a curved profile designed to follow the natural contour of the breast, can be incorporated and tracked within the imaging system 108 using the removable tracking sensor 118 configurations.

The imaging system 108 can provide patient-specific scan data for use in finite element analysis (FEA) and computational fluid dynamics (CFD) simulations of treatment outcomes. When pre-operative CT or MRI data is available from the imaging system 108, the system 100 can generate three-dimensional models of the body and relevant tissue types through segmentation. By applying finite element analysis or computational fluid dynamics to these three-dimensional models, the system 100 can simulate treatment outcomes and predict energy delivery patterns based on the position of the treatment head 114 relative to the patient's anatomy before actual treatment delivery. Simulation results can incorporate tissue growth and decline responses, transport phenomena, metabolic behavior, and varying therapeutic concentrations as applicable to the treatment modality.

Simulation results can be displayed in three dimensions through the augmented reality visualization system 102, providing physicians with holographic visualization of predicted treatment effects in the patient coordinate space. Simulations can alternatively be used to inform the probe placement algorithm to suggest optimal treatment head 114 placement based on predicted energy delivery patterns. These simulations can be performed pre-operatively using planning data or in real time during the procedure to guide treatment delivery. The registration tracking system 104 can maintain continuous alignment between simulation results and physical system components through the tracking sensors 118, enabling accurate spatial correspondence between simulated and actual treatment delivery throughout the procedure.

The system 100 can support procedural operation across three distinct phases: localization, planning, and treatment. Each phase can involve coordinated operation of the augmented reality visualization system 102, the registration tracking system 104, the treatment delivery system 106, and the imaging system 108, with each system contributing capabilities specific to the needs of that phase. The three phases can be executed sequentially or can overlap depending on procedural requirements, and the system 100 can provide phase-specific guidance and visualization through the head-mounted device 110 at each stage. The solutions described across phases are not mutually exclusive and can be combined or interchanged depending on the procedural scenario and the specific configuration of equipment available.

During the localization phase, the physician can use the diagnostic ultrasound probe 116 to locate and identify target tissue under real-time ultrasound imaging. The augmented reality visualization system 102 can display the acoustic beam geometry of the diagnostic ultrasound probe 116 as a holographic flashlight projected in the patient coordinate space by registering the head-mounted device 110 coordinate space to the treatment delivery system 106 through tracking sensors 118 mounted on the robotic arm 112 or treatment head 114. This ultrasound flashlight visualization can provide direct contextual display of the real-time ultrasound image relative to the physical patient as viewed through the head-mounted device 110, without requiring the physician to correlate a two-dimensional screen image with the three-dimensional patient space.

The imaging system 108 can provide multi-planar viewing with rotating and translating diagnostic ultrasound capabilities to support full volumetric targeting of the region of interest. Where pre-operative CT, MRI, or other medical imaging is available, the imaging system 108 can enable fusion of that imaging with the real-time ultrasound by relating the imaging coordinate system to the coordinate system of the tracked ultrasound beam. This registration can also be performed manually by aligning a segmented skin surface mesh extracted from pre-operative imaging with the patient's actual skin surface as viewed through the head-mounted device 110. The probe placement algorithm can be applied during localization to suggest optimal placement locations for the diagnostic ultrasound probe 116 and treatment head 114 prior to scout scanning, reducing the time required to identify the target under ultrasound.

Once the target tissue is identified, the system 100 can stamp and save the position and orientation of the treatment head 114 or freehand diagnostic probe in six degrees of freedom. This position stamp can enable the treatment head 114 to return to the same location and orientation after the coupling membrane is applied and the robotic arm 112 is repositioned, resolving the previously disjointed transition between localization and treatment phases. The system 100 can also stamp the position and orientation of a freehand diagnostic probe used during localization, enabling the robotic arm 112 to subsequently guide the treatment head 114 to that same position. A holographic light ray can be displayed extending from the treatment head 114 or probe head to represent the stamped trajectory and facilitate manual or robotic realignment.

During the planning phase, the system 100 can overlay and fuse the planned three-dimensional treatment zone with the ultrasound beam hologram using the registration established during the localization phase. The treatment delivery system 106 can enable adjustment of the size, shape, and location of planned treatment volumes with target and margin contours, and visual representations of these contours can be overlaid onto the real-time diagnostic ultrasound from the imaging system 108 through the head-mounted device 110. This three-dimensional volumetric visualization can provide planning utility not available on two-dimensional screens, enabling the physician to evaluate the planned treatment zone in context with the patient's actual anatomy.

Where holographic image-based anatomy is provided through registration of pre-operative imaging to the patient coordinate space, the system 100 can enable visualization of target tissue and surrounding structures in context with the ultrasound flashlight, highlighting protected structures to avoid and facilitating margin assessment. Protected structures can be marked during the planning phase in CT or segmentation data through the imaging system 108. If the planned treatment delivery zone overlaps with any marked protected structures, the system 100 can highlight the structure, display warnings through the head-mounted device 110, or disable the treatment delivery system 106 to prevent unintended energy delivery to protected tissue. Marked protected structures can also inform the probe placement algorithm to ensure suggested placement locations provide clear paths for energy delivery while avoiding marked structures.

The system 100 can utilize the inertial measurement unit 120 on the robotic arm 112 to maintain registration accuracy during planning. If the robotic arm 112 or treatment head 114 is moved or bumped during the planning phase, the inertial measurement unit 120 can trigger re-registration between system components before planning is continued or treatment is initiated. The system 100 can also allow determination of precise energy requirements for target destruction through measurements at discrete points within each target, with these measurements used to determine treatment parameters for automated delivery.

During the treatment phase, the treatment delivery system 106 can provide fully automated dynamic movement of the treatment head 114 through optimized patterns covering the planned treatment volumes. The physician can initiate automated treatment after confirming system registration, target localization, and treatment zone planning. The system 100 can provide continuous real-time monitoring of imaging and treatment status through the head-mounted device 110 throughout automated delivery.

Where holographic image-based anatomy is provided, the system 100 can fine-adjust the holographic anatomy to align with the target under real-time ultrasound prior to treatment delivery. As the treatment pattern progresses along its planned path, the system 100 can decimate or deform the holographic anatomy mesh to show the expected tissue effects of the treatment, providing conceptual real-time feedback about treatment progress even when the active treatment zone is difficult to visualize directly under diagnostic ultrasound. Where the active treatment zone is detectable under live ultrasound or through other detection means, that information can be fed into the system 100 to show the location of the treatment zone as it is produced anatomically, and to detect internal anatomy movement during the procedure that may indicate mis-targeting.

The time-of-flight camera 122 can track the motion of the patient's chest wall or abdomen during breathing to provide respiratory motion compensation throughout the treatment phase. By marking the target location at the peak and trough of the breathing cycle using the diagnostic ultrasound probe 116, the system 100 can determine the target position at the extremes of the respiratory cycle. Using this information together with real-time respiratory phase tracking from the time-of-flight camera 122, the system 100 can interpolate the target location at any given time point throughout the respiratory cycle. The robotic arm 112 and treatment head 114 can compensate for respiratory motion by delivering treatment to the interpolated target location continuously, or the system 100 can gate treatment delivery to specific respiratory phases. Where the augmented reality visualization system 102 is registered to the respiration tracking system, the holographic representation of the target can be updated in real-time to reflect tracked respiratory motion, providing visual confirmation that the holographic guidance remains aligned with the moving target.

Safety interlocks can remain active throughout the treatment phase. The inertial measurement unit 120 can disable treatment delivery if motion of the robotic arm 112 is detected. The system 100 can disable treatment delivery if the physician's gaze is detected to be directed away from the treatment zone. The planned treatment delivery zone can be continuously compared against marked protected structures, and treatment can be halted if overlap is detected. Treatment delivery can resume only after the physician confirms re-registration or confirms that treatment zone alignment is maintained.

The system 100 can enable marking of protected structures, which are anatomical features such as nearby tissues and organs that require protection from unintended energy delivery during the procedure. Protected structures can be marked during the planning phase in CT imaging, segmentation data, or other imaging data from the imaging system 108. Marking can be performed manually by the physician or through automated segmentation methods including active contouring or artificial intelligence-based anatomical feature detection. The registration tracking system 104 can maintain continuous alignment between the marked protected structures and physical system components through tracking sensors 118, ensuring accurate spatial representation of protected areas relative to the treatment zone throughout all procedural phases.

When protected structures are marked, the system 100 can continuously compare the planned treatment delivery zone against all marked structures. If the treatment delivery zone from the treatment delivery system 106 overlaps with any marked protected structure, the system 100 can highlight the overlapping structure through the head-mounted device 110, display warnings to the physician, or automatically disable treatment delivery to prevent unintended energy delivery to protected tissue. Treatment delivery can resume only after the physician confirms that the overlap has been resolved or that treatment zone alignment is maintained. Marked protected structures can also be provided as an input to the probe placement algorithm, informing suggested placement locations to ensure that energy delivery paths avoid marked structures.

The system 100 can incorporate a gaze-based safety interlock configured to automatically disable treatment delivery when the physician is not actively monitoring the treatment zone through the head-mounted device 110. The gaze-based interlock can use the gaze tracking capabilities of the head-mounted device 110 to determine whether the physician's line of sight is directed toward the treatment zone during active energy delivery. If the physician's gaze is detected to be directed away from the treatment zone for a defined duration, the treatment delivery system 106 can be automatically disabled. Treatment delivery can resume when the physician's gaze is returned to the treatment zone and the physician confirms readiness to proceed.

The system 100 can incorporate a motion-based safety interlock using the inertial measurement unit 120 mounted on the robotic arm 112 or treatment head 114. The inertial measurement unit 120 can function as a fail-safe component configured to detect changes in position or rotation from a previously registered configuration rather than as a standalone absolute position tracking device. If the inertial measurement unit 120 detects motion of the robotic arm 112 or treatment head 114 exceeding a defined threshold during active treatment delivery, the treatment delivery system 106 can be automatically disabled to prevent unintended energy delivery to tissue outside the planned treatment zone. Treatment delivery can resume only after the physician confirms re-registration of the system or confirms that treatment zone alignment is maintained within the registration tracking system 104.

The gaze-based interlock, motion-based interlock, and protected structure overlap detection can operate simultaneously and independently, with any one interlock condition sufficient to disable treatment delivery. The system 100 can be configured to require explicit physician confirmation to resume treatment delivery after any safety interlock is triggered. Interlock events can be recorded by the data storage and management capabilities of the registration tracking system 104, enabling post-procedure review of any conditions that caused interruptions to automated treatment delivery.

The safety interlocks can apply across all therapeutic modalities supported by the treatment delivery system 106 and can operate during both manual and automated treatment delivery phases. The combination of protected structure marking, gaze-based monitoring, and motion detection can provide a layered safety architecture that addresses the primary risk conditions of mis-targeting, inattention, and undetected system displacement during treatment delivery.

The system 100 can incorporate respiratory motion compensation to maintain accurate spatial alignment between the treatment head 114 and the target tissue throughout the breathing cycle. Target tissue in the abdomen and thorax, including the liver, kidneys, and other organs, can move significantly between the peak and trough of each breathing cycle. Without compensation, this motion can cause the therapeutic energy delivered by the treatment head 114 to miss the intended target tissue or interact with adjacent protected structures. The system 100 can support multiple respiratory motion compensation approaches, implemented individually or in combination.

The system 100 can use the time-of-flight camera 122 mounted on the treatment head 114 to directly measure motion of the patient's chest wall or abdomen surface during breathing. The time-of-flight camera 122 can generate a real-time depth map of the patient's surface and can track the displacement of that surface through the respiratory cycle, producing a continuous signal representing the patient's respiratory phase and amplitude. This surface motion signal can be used as a surrogate for the internal position of the target tissue throughout the breathing cycle.

The time-of-flight camera 122 can be integrated with the registration tracking system 104 directly or through other tracking sensors 118 to correlate surface motion data with the registered coordinate system of the treatment delivery system 106 and head-mounted device 110. When the time-of-flight camera 122 is mounted on the treatment head 114 and the treatment head 114 is itself registered within the common coordinate system, the respiratory motion measurements produced by the time-of-flight camera 122 can be directly related to the target coordinate system without requiring additional calibration steps.

The system 100 can enable the physician to mark the position of the target tissue using the diagnostic ultrasound probe 116 at both the peak and trough of the breathing cycle. At the peak of inhalation, the physician can use the diagnostic ultrasound probe 116 to locate and mark the target tissue position in the registered coordinate system of the head-mounted device 110. At the trough of exhalation, the physician can repeat this marking process to capture the target position at the opposite extreme of the respiratory cycle. Target marking can be performed manually by the physician or through automatic methods including active contouring or artificial intelligence-based detection of the target boundary within the diagnostic ultrasound image.

By establishing the known target position at both extremes of the breathing cycle, the system 100 can define the range of target motion attributable to respiration. Using the real-time respiratory phase signal derived from the time-of-flight camera 122 or other respiratory tracking modality, the system 100 can interpolate the current target position at any point within the breathing cycle between the two marked extremes. This interpolated target position can be provided to the treatment delivery system 106 to enable continuous or gated respiratory compensation during energy delivery.

Using the interpolated target position, the system 100 can direct the robotic arm 112 to continuously adjust the position of the treatment head 114 to follow the target tissue through the respiratory cycle. This continuous robotic motion compensation can allow therapeutic energy delivery to proceed without interruption throughout breathing, maintaining consistent alignment between the treatment head 114 and the moving target at all points in the respiratory cycle. Alternatively, the system 100 can implement respiratory gating, where treatment delivery is paused and resumed based on the patient's respiratory phase. In a gated configuration, the treatment delivery system 106 can deliver energy only when the patient's respiratory phase falls within a defined window around a selected target phase, such as end-exhalation, and can automatically pause treatment delivery when the patient breathes outside that window. The system 100 can support both continuous motion compensation and respiratory gating configurations, and can switch between them based on procedural requirements and target tissue characteristics.

When the augmented reality visualization system 102 is registered to the respiratory motion tracking system, the holographic representation of the target tissue displayed through the head-mounted device 110 can be updated in real time to reflect the tracked respiratory motion. As the patient breathes, the holographic target display can move correspondingly to show the current interpolated position of the target tissue, providing the physician with continuous visual confirmation that the holographic guidance remains spatially aligned with the actual moving target. This real-time holographic update can provide visual feedback during both the planning phase and the treatment phase, enabling the physician to observe and confirm the magnitude and pattern of respiratory target motion before committing to a compensation strategy.

When the diagnostic ultrasound probe 116 is operational during treatment delivery, the system 100 can leverage the real-time ultrasound data for additional motion tracking capabilities beyond surface-based time-of-flight sensing. Ultrasound-based motion tracking can detect internal anatomy displacement, including movement of the target tissue itself rather than only the surface surrogate used by the time-of-flight camera 122. When internal anatomy movement is detected during the treatment phase, this information can be fed into the system 100 to update the interpolated target position, provide alerts to the physician, or trigger pausing of treatment delivery to prevent mis-targeting. Ultrasound-based motion tracking can operate in conjunction with time-of-flight surface tracking through sensor fusion to produce a more robust and accurate respiratory compensation signal than either modality alone.

In addition to time-of-flight sensing and ultrasound-based tracking, the system 100 can support alternative or supplementary respiratory tracking modalities. Optical tracking sensors 118 can track surface markers placed on the patient's chest wall or abdomen to measure respiratory motion. Inertial measurement units 120 can provide motion data supplementary to surface sensing when mounted on components in contact with or near the patient. Patient monitoring systems providing physiological respiratory signals, such as respiratory bellows or spirometry, can be integrated with the system 100 as alternative respiratory phase inputs. The system 100 can combine data from any combination of these modalities through sensor fusion to produce a respiratory compensation signal with improved robustness and reliability relative to any single modality.

The augmented reality visualization system 102 can enable different clinical team members to view role-specific holographic projections through their respective head-mounted devices 110. The system 100 can support multiple users operating simultaneously in different roles, with each user's head-mounted device 110 displaying content tailored to their specific procedural responsibilities. The system 100 can establish distinct role categories, including operator and spectator roles, with each role having access to specific types of information and control capabilities through their respective head-mounted device 110.

In a typical procedural configuration, a table-side assistant can monitor the physical equipment, the patient, and the treatment zone while viewing holographic projections of the ultrasound beam, medical imaging, and targeting and positioning information through their head-mounted device 110. A console operator can focus on controlling the robotic arm 112 and the treatment delivery system 106 while monitoring treatment delivery parameters through their head-mounted device 110 or console display. Additional team members such as anesthesia or nursing staff can receive simplified status displays providing relevant procedural information without full guidance overlays. Each role-based display can be configured independently with respect to content type, visualization parameters, and interaction modalities based on the responsibilities associated with that role. The role-specific visualization architecture of the system 100 can extend beyond traditional head-mounted device 110 configurations. The console display of the treatment delivery system 106 can be implemented as an augmented reality or mixed reality display, enabling the console operator to view holographic content integrated with the physical console interface. This display can present role-appropriate procedural information including treatment delivery parameters, robotic arm 112 positioning data, and treatment zone status in a format optimized for console-based operation.

The system 100 can enable a shared spatial experience in which multiple team members simultaneously view holographic content that is spatially registered to the same physical coordinate system, allowing all team members to see the same holographic representations of the ultrasound beam, treatment zone, and anatomy from their respective positions in the room. The registration tracking system 104 can maintain continuous spatial registration for all active head-mounted devices 110 simultaneously, ensuring that holographic content displayed through each device is accurately aligned with the patient and the treatment delivery system 106 from the perspective of each user's individual viewpoint. This shared spatial experience can facilitate real-time communication and coordination between team members during the localization, planning, and treatment phases without requiring team members to reference external screens or verbally describe spatial relationships.

The system 100 can support remote visualization configurations in which a physician or specialist who is not physically present in the procedural environment can receive procedural information through a networked connection to a remote head-mounted device 110 or other display. In remote configurations, the remote user can view holographic representations of the treatment zone, imaging data, robotic arm 112 positioning, and real-time treatment status. The remote user can provide guidance, approve treatment parameters, or perform a virtual sign-off on treatment initiation through the remote display interface. Remote visualization can enable procedural support in outpatient, mobile, or distributed clinical settings where on-site specialist presence is not available or required.

The remote visualization capability can extend the role-based framework to remote participants, enabling a remote specialist to assume an operator or spectator role with corresponding access to information and control capabilities. The system 100 can support remote participation in each procedural phase, enabling a remote physician to review target localization and treatment zone planning before authorizing treatment delivery initiation. The remote user can interact with holographic content through the same gesture, voice, and gaze-based interaction modalities available to on-site users through their head-mounted device 110. Remote visualization configurations can be applicable to any therapeutic modality supported by the treatment delivery system 106 and can operate across standard healthcare network infrastructure.

The system 100 can integrate artificial intelligence and automated computing capabilities across all four subsystems to enhance workflow efficiency, reduce procedural complexity, and improve consistency of outcomes. Artificial intelligence capabilities can be applied to image analysis, anatomical feature detection and segmentation, target marking and contouring, scan quality assessment, and treatment planning optimization. Automated computing capabilities can be applied to probe placement suggestion, sensor fusion, kinematic solving, respiratory motion interpolation, and treatment delivery pattern execution. These capabilities can operate individually or in combination across the localization, planning, and treatment phases described herein.

Automated segmentation can be applied to diagnostic ultrasound images, CT data, MRI data, or fused multi-modal imaging datasets to identify target tissues, organ boundaries, vessel structures, and protected structures without requiring manual contouring by the physician. In a semi-automated configuration, the physician can indicate a seed point within the target tissue and the system 100 can automatically expand a contour from that seed point to detect and delineate the target boundary. In a fully automated configuration, artificial intelligence algorithms can detect the target boundary directly without requiring physician input beyond confirming the result. Segmented structures can be rendered as three-dimensional surface meshes and displayed as holographic overlays through the head-mounted device 110 in the patient coordinate space. Artificial intelligence and automated computing capabilities can inform the probe placement algorithm, the respiratory motion compensation capabilities, the simulation capabilities, and the automated treatment delivery capabilities.

The registration tracking system 104 can include data storage and management capabilities configured to record procedural data throughout all phases of the procedure. Stored data can include pre-operative planning data, coordinate transformations established during registration, real-time ultrasound image streams from one or more diagnostic ultrasound probes 116 with associated position and orientation data from tracking sensors 118, robotic arm 112 positioning data, treatment delivery parameters, respiratory motion tracking data from the time-of-flight camera 122, inertial measurement unit 120 motion logs, and simulation results from finite element analysis and computational fluid dynamics. Coordinate transformations can be stored alongside procedural data to preserve the spatial relationships between coordinate systems as they existed at each point during the procedure.

The system 100 can stamp and save planned therapeutic volumes (PTVs), treatment head 114 positions and orientations, and procedural parameters throughout the localization, planning, and treatment phases. Stamped PTVs can be linked to or compared with treatment monitoring data to analyze therapy delivery outcomes in real time or post-treatment. Data storage can reside within the head-mounted device 110, on an external computing device communicating via a network, or on a remote server or cloud-based storage system. Access to stored procedural data can be controlled through authentication mechanisms to maintain patient data privacy and comply with applicable healthcare data security requirements.

Stored procedural data can be retrieved and replayed to reconstruct the procedural guidance view at any point during or after the procedure. The system 100 can record the planned therapeutic volume and compare it to the delivered treatment zone during or after the procedure to assess margin coverage and identify areas of potential under-treatment or critical structure proximity. Retrospective review can support post-procedure assessment of treatment zone coverage, instrument trajectory accuracy, and quality assurance. The ability to link stamped PTVs with treatment monitoring data and simulation results can be particularly valuable for determining therapy efficacy and evaluating outcomes across patient populations over time.

### EXAMPLES

With reference to the several figures enclosed herewith, example embodiments of the present technology are described below. The following examples are intended to illustrate specific configurations of the system 100 and are not intended to limit the scope of the present disclosure. The solutions described in the following examples are not mutually exclusive and can be combined or interchanged as described in the detailed description above.

With reference to FIG. 1, the system 100 is shown in an overview configuration illustrating the four primary subsystems and their relationships. The augmented reality visualization system 102, which can include a head-mounted device 110, is shown in spatial relation to the treatment delivery system 106, which includes a robotic arm 112 supporting a treatment head 114. The treatment head 114 integrates a diagnostic ultrasound probe 116 and is equipped with one or more tracking sensors 118 and a time-of-flight camera 122. The registration tracking system 104 is shown as the coordinating subsystem that receives tracking data from sensors 118 mounted on the robotic arm 112, treatment head 114, and other system components, and transforms these data into a common coordinate system shared by the head-mounted device 110 and treatment delivery system 106. The imaging system 108 is shown as a connected subsystem providing both real-time diagnostic ultrasound data from the probe 116 integrated with the treatment head 114 and pre-operative imaging data from CT, MRI, or other tomographic modalities.

During localization, the physician uses the diagnostic ultrasound probe 116 to locate and identify target tissue, with the registration tracking system 104 enabling the augmented reality visualization system 102 to display the ultrasound beam as a holographic overlay relative to the patient. During planning, the system 100 overlays planned treatment zones and anatomical structures 126 from pre-operative imaging in the patient coordinate space. During treatment, the treatment delivery system 106 provides automated dynamic movement of the treatment head 114 through planned treatment volumes while the imaging system 108 and registration tracking system 104 maintain continuous spatial registration and monitoring of the therapeutic energy beam 124.

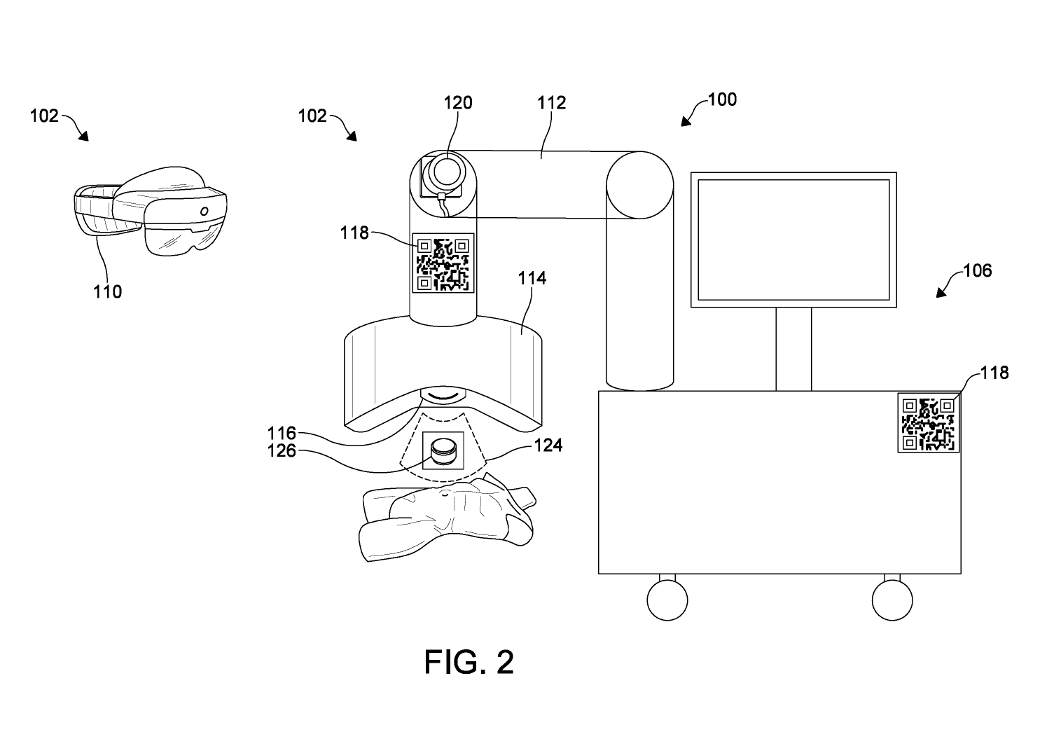

With reference to FIG. 2, the system 100 is shown in two registration configurations that do not require pre-operative imaging data, relying instead on tracking sensors 118 and system interface data to establish and maintain spatial registration between the augmented reality visualization system 102 and the treatment delivery system 106. The two configurations are shown together in FIG. 2 to illustrate the range of sensor placement strategies available when image-based anatomy is not used.

In the first configuration, tracking sensors 118 are mounted on the robotic arm 112 and/or directly on the treatment head 114. The registration tracking system 104 uses data from these sensors to transform the coordinate systems of the treatment delivery system 106 and the head-mounted device 110 into a common coordinate system, enabling holographic display of the live ultrasound beam and pre-planned treatment zone without anatomical image overlay. In the second configuration, a tracking sensor 118 is mounted on the main unit of the treatment delivery system 106 rather than on the robotic arm 112 or treatment head 114. In this configuration, the registration tracking system 104 utilizes known robotic linkage geometry and reported joint settings from the treatment delivery system 106 to determine the position and orientation of the treatment head 114 through kinematic solving rather than by directly tracking the treatment head 114 with a sensor. This configuration can be advantageous when direct mounting of sensors 118 on the robotic arm 112 or treatment head 114 is not available or practical.

FIG. 2 illustrates the role of the inertial measurement unit 120 mounted on the robotic arm 112 as a fail-safe component in both configurations. Because no image-based registration anchor is available to detect positional drift or unintended movement, the inertial measurement unit 120 provides continuous monitoring of the robotic arm 112 position and rotation. If the inertial measurement unit 120 detects motion exceeding a defined threshold during active treatment, the treatment delivery system 106 can be automatically disabled and the system 100 can prompt the physician to confirm re-registration before treatment resumes.

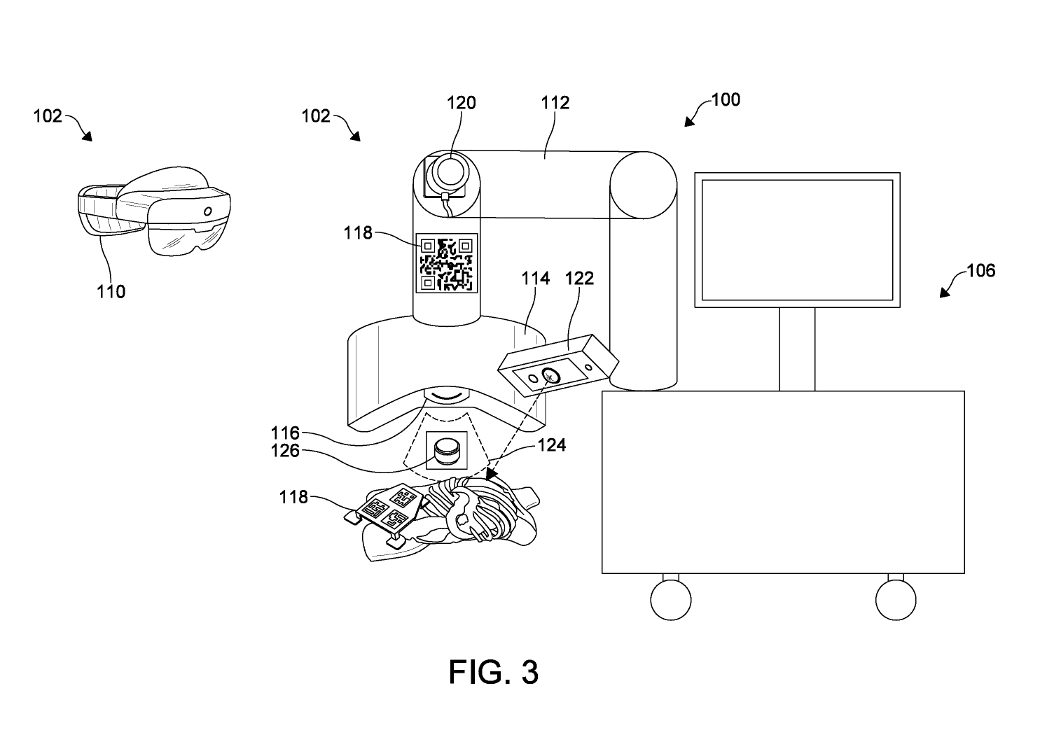

With reference to FIG. 3, the system 100 is shown in a registration configuration that incorporates pre-operative CT, MRI, or other medical imaging data alongside real-time diagnostic ultrasound from the imaging system 108. Tracking sensors 118 are mounted on the robotic arm 112 and/or treatment head 114, and an additional tracking sensor 118 is mounted on or associated with the patient, enabling the registration tracking system 104 to detect and compensate for patient movement relative to the treatment delivery system 106 during the procedure.

FIG. 3 illustrates the two methods by which the imaging system 108 can register pre-operative imaging to the patient coordinate space. In the first method, multi-planar reconstruction slices from CT or MRI are adjusted to match the stationary real-time ultrasound image acquired by the diagnostic ultrasound probe 116, relating the imaging coordinate system to the coordinate system of the tracked ultrasound beam. In the second method, a skin surface mesh segmented from the pre-operative imaging data is displayed as a hologram through the head-mounted device 110, and the physician manually translates and rotates the holographic mesh until it aligns with the patient's actual skin surface as viewed through the head-mounted device 110. Once registered, the system 100 can display holographic three-dimensional anatomical structures from pre-operative imaging overlaid on the patient in the patient coordinate space, providing contextual visualization of the target tissue, surrounding anatomy, and protected structures alongside the real-time ultrasound beam.

FIG. 3 also shows the time-of-flight camera 122 mounted on the treatment head 114 as a dashed element, indicating that respiratory motion compensation using the time-of-flight camera 122 is an optional addition to the image-based anatomy registration configuration. When included, the time-of-flight camera 122 generates a real-time depth map of the patient's surface and tracks respiratory displacement to produce a continuous respiratory phase signal, which the system 100 uses together with target positions marked at the breathing extremes to interpolate the current target position throughout the breathing cycle.

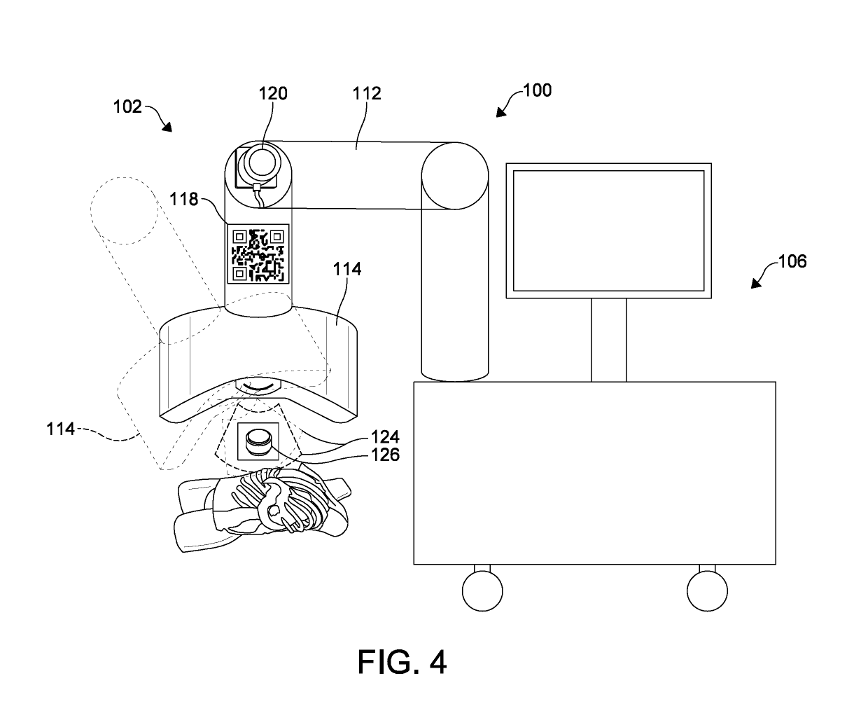

With reference to FIG. 4, the system 100 is shown in a configuration in which the probe placement algorithm is used to suggest optimal placement locations for the treatment head 114 and diagnostic ultrasound probe 116 based on pre-operative imaging data from the imaging system 108 and the identified target tissue location. FIG. 4 illustrates the rotated and repositioned treatment head 114 and robotic arm 112 configuration that results from applying the probe placement algorithm's suggestions, showing how the algorithm can recommend positions that differ substantially from a default starting configuration in order to optimize acoustic access to the target tissue.

FIG. 4 illustrates how the probe placement algorithm accepts multiple inputs including tissue type information derived from the imaging data, acoustic pathway characteristics, coupling fluid parameters, probe type specifications, and the locations of any marked protected structures, and processes these inputs to generate a ranked set of suggested placement locations. Each suggested placement location is validated against the mechanical constraints of the robotic arm 112 through the registration tracking system 104 before being presented to the physician, ensuring that all suggested positions are physically achievable. The system 100 can save multiple placement locations simultaneously, for example, one position optimized for diagnostic imaging and a separate position optimized for therapeutic energy delivery, enabling the physician to switch between saved positions as needed during the procedure.

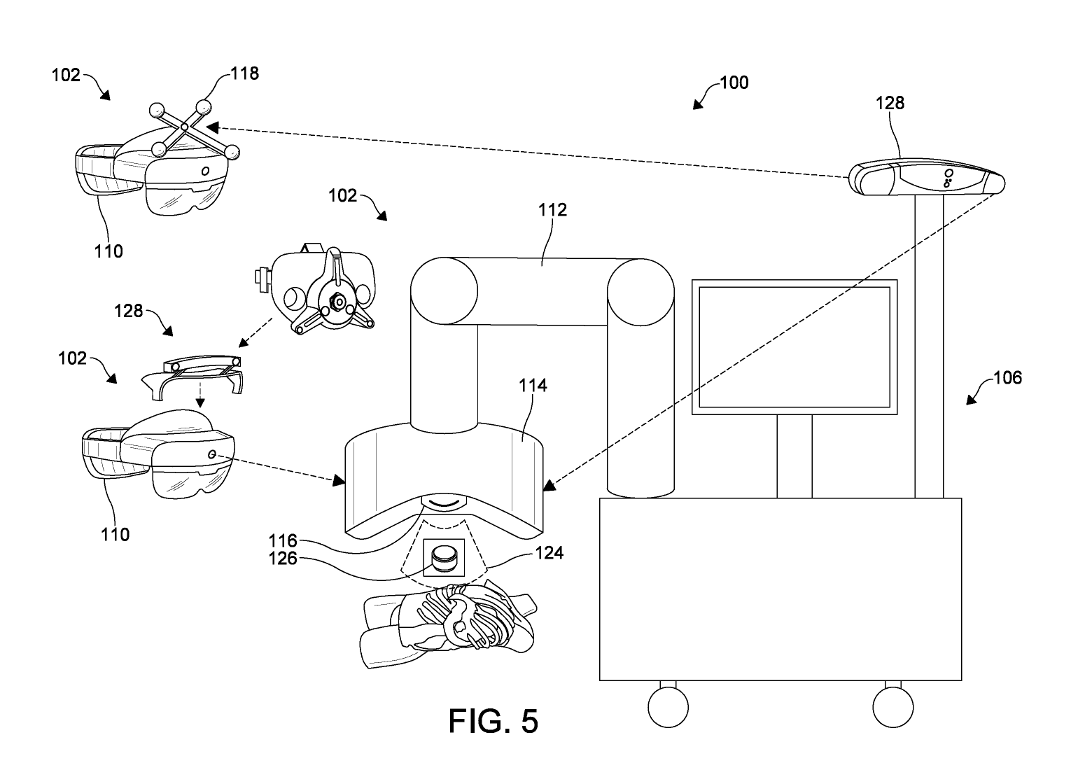

With reference to FIG. 5, the system 100 is shown in alternative tracking configurations applicable when the treatment delivery system 106 does not have an internal coordinate system available to the registration tracking system 104. FIG. 5 illustrates two primary alternative tracking approaches. In the first, an external optical tracking system 128 tracks reflective or active optical markers mounted on the treatment delivery system 106, including the robotic arm 112 and treatment head 114, and optionally on the head-mounted device 110. The optical tracking system simultaneously observes all tracked components and transforms their positions and orientations into a common coordinate system, enabling the registration tracking system 104 to maintain continuous spatial alignment between the augmented reality visualization system 102 and the treatment delivery system 106 without requiring electromagnetic tracking infrastructure. If the head-mounted device 110 can access the optical tracking system's coordinate output directly through a software interface, an optical marker on the head-mounted device 110 is not required.

In the second configuration shown in FIG. 5, infrared, acoustic, or electromagnetic tracking sensors 118 are used in place of or in addition to optical tracking. Infrared or acoustic sensors 118 can be mounted on the head-mounted device 110 and directed toward the treatment delivery system 106, enabling the head-mounted device 110 to directly track the treatment head 114 and robotic arm 112. Alternatively, an electromagnetic tracking system can be positioned beneath or beside the patient, enabling tracking of both the patient and the diagnostic ultrasound probe 116 equipped with an electromagnetic tracking sensor 118. This electromagnetic patient-area configuration enables the two-dimensional ultrasound image from the diagnostic ultrasound probe 116 to be precisely located within the coordinate system of the augmented reality visualization system 102, and also enables detection of patient movement through the patient-area sensor.

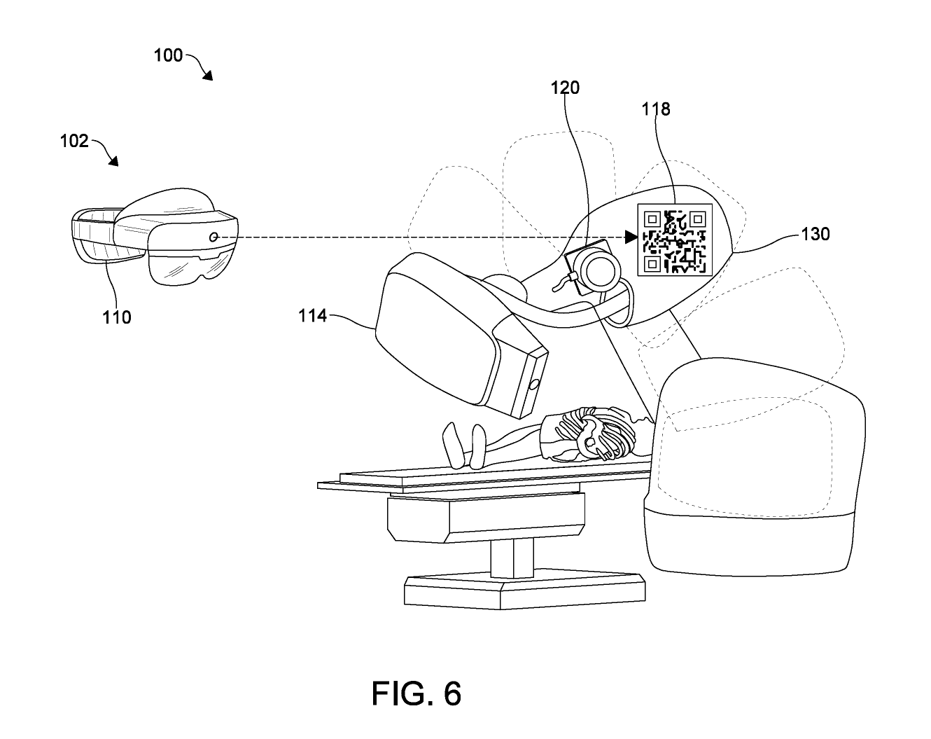

With reference to FIG. 6, the system 100 is shown in a radiotherapy application in which the treatment delivery system 106 is a radiotherapy system 130, such as a CyberKnife or GammaKnife system, in which the robotic arm 112 supports a radiotherapy treatment head 114. Tracking sensors 118 are mounted on one or more locations on the robotic arm 112 and treatment head 114, with each component individually tracked to enable holographic projection of the pre-planned treatment zone relative to the patient's anatomy through the head-mounted device 110.

FIG. 6 illustrates how the system 100 applies the same registration, visualization, and safety architecture described throughout the detailed description to a non-ultrasound therapeutic modality. The registration tracking system 104 can utilize positional and rotational information from the radiotherapy system's own coordinate interface, where available, to determine the position of the treatment head 114 through kinematic solving. Where pre-operative CT or MRI imaging is available, the imaging system 108 can project that imaging data into the patient coordinate space by relating multi-planar images to the treatment head 114 position established through the tracking sensors 118, or by manually aligning a segmented skin surface mesh with the patient's actual skin surface. The inertial measurement unit 120 mounted on the robotic arm 112 can trigger re-registration or disable therapeutic delivery if motion is detected during treatment, consistent with the safety architecture. FIG. 6 thus illustrates that the registration, visualization, and control capabilities of the system 100 are applicable to any capital equipment system involving a robotic linkage between a base unit and a therapeutic delivery tool.

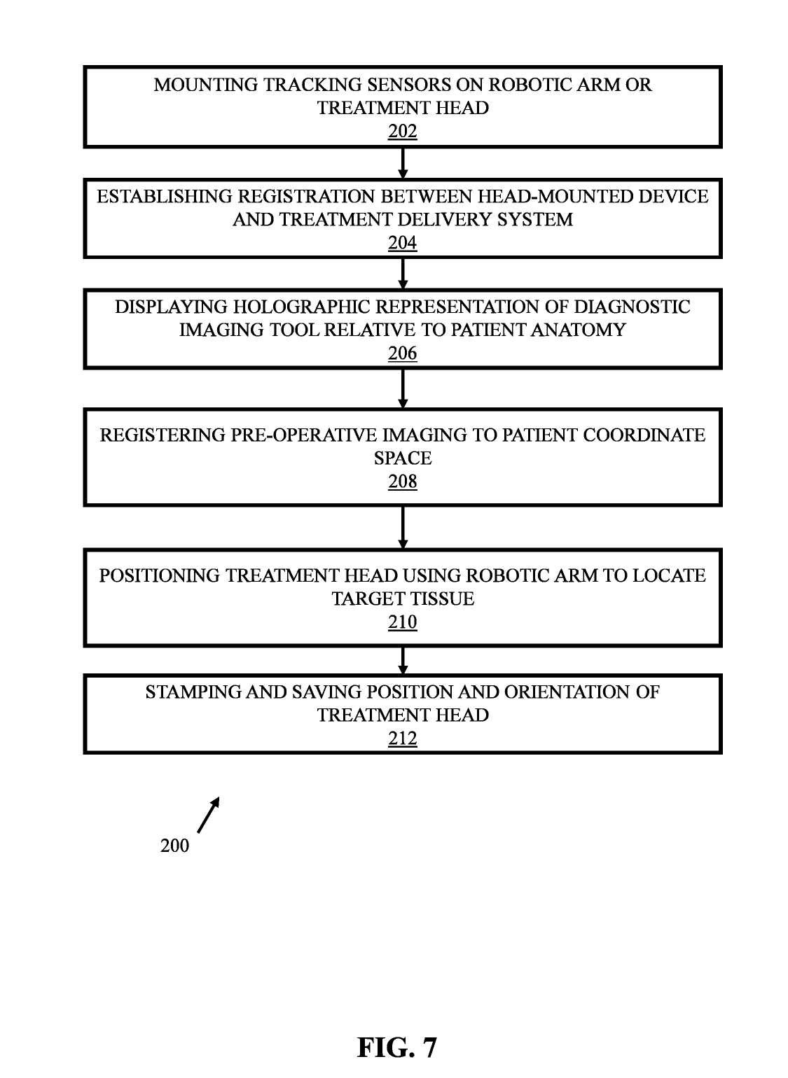

With reference to FIG. 7, a method 200 for localizing a target tissue under holographic augmented reality guidance using the system 100 can include the following steps. Method 200 can include a step 202 of mounting one or more tracking sensors 118 on the robotic arm 112, treatment head 114, or main unit of the treatment delivery system 106 and donning the head-mounted device 110. Method 200 can include a step 204 of initiating registration between the head-mounted device 110 and the treatment delivery system 106 through the registration tracking system 104, establishing a common coordinate system shared by the augmented reality visualization system 102 and the treatment delivery system 106. As illustrated in FIG. 7, registration can be established through direct tracking of sensors 118 mounted on the robotic arm 112 or treatment head 114, through kinematic solving from a sensor 118 mounted on the main unit of the treatment delivery system 106, or through an external optical, infrared, acoustic, or electromagnetic tracking system.

Method 200 can include a step 206 of displaying a holographic representation of the ultrasound beam of the diagnostic ultrasound probe 116 as a flashlight relative to the probe position through the head-mounted device 110 upon establishing registration, providing direct contextual visualization of the real-time ultrasound image relative to the patient. Method 200 can include a step 208 of optionally registering pre-operative CT or MRI imaging data from the imaging system 108 to the patient coordinate space by adjusting multi-planar reconstruction slices to match the stationary real-time ultrasound image, or by manually aligning a segmented holographic skin surface mesh with the patient's actual skin surface through the head-mounted device 110, and displaying three-dimensional holographic anatomical structures overlaid on the patient. Method 200 can include a step 210 of positioning the treatment head 114 using the robotic arm 112 to locate the target tissue under the holographically displayed ultrasound beam, with the augmented reality visualization system 102 providing continuous spatial context through the head-mounted device 110. Method 200 can include a step 212 of stamping and saving the position and orientation of the treatment head 114 in six degrees of freedom through the registration tracking system 104 once the target tissue has been located, with the augmented reality visualization system 102 displaying a holographic light ray projecting from the treatment head 114 along the stamped trajectory as a persistent spatial reference in the patient coordinate space.

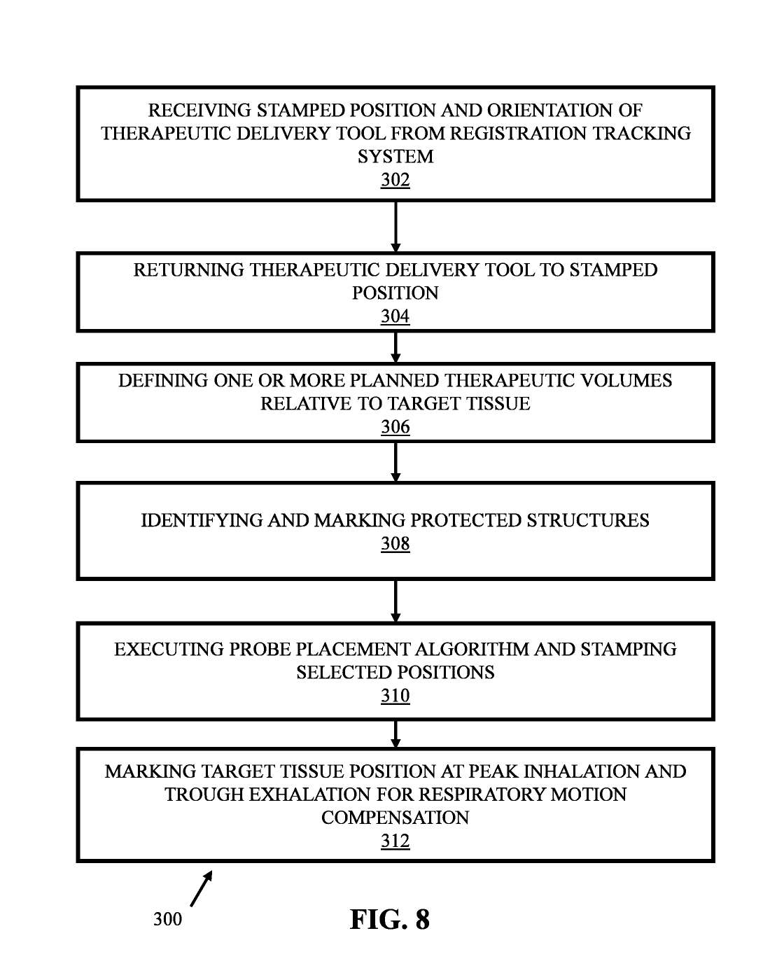

With reference to FIG. 8, a method 300 for planning a non-contacting energy ablation procedure under holographic augmented reality guidance using the system 100 can include the following steps. Method 300 can include a step 302 of receiving the stamped position and orientation of the treatment head 114 from the registration tracking system 104 following completion of the localization phase described in Method 200 above, and directing the robotic arm 112 to move the treatment head 114 away from the patient to permit application of the coupling membrane to the treatment head 114. Method 300 can include a step 304 of returning the treatment head 114 to the stamped position after the coupling membrane has been applied, with the augmented reality visualization system 102 displaying the offset between the current position of the treatment head 114 and the stamped target position through the head-mounted device 110 to confirm accurate repositioning, as illustrated in FIG. 8.

Method 300 can include a step 306 of defining one or more planned therapeutic volumes relative to the target tissue identified during the localization phase, with the augmented reality visualization system 102 displaying the planned treatment zone as a holographic overlay in the patient coordinate space through the head-mounted device 110, as illustrated in FIG. 8. Method 300 can include a step 308 of optionally identifying and marking protected structures within the pre-operative imaging data from the imaging system 108, either manually by the physician or through automated anatomical segmentation using artificial intelligence-based detection, and integrating the marked protected structures with the planned treatment zone to enable continuous overlap monitoring. Method 300 can include a step 310 of optionally executing the probe placement algorithm above to generate a ranked set of validated treatment head 114 placement suggestions, selecting one or more suggested positions, and stamping the selected positions through the registration tracking system 104. Method 300 can include a step 312 of optionally marking the position of the target tissue at the peak of inhalation and the trough of exhalation using the diagnostic ultrasound probe 116, establishing the spatial range of target motion attributable to respiration in the registered coordinate system of the head-mounted device 110 in preparation for respiratory motion compensation during the treatment phase.

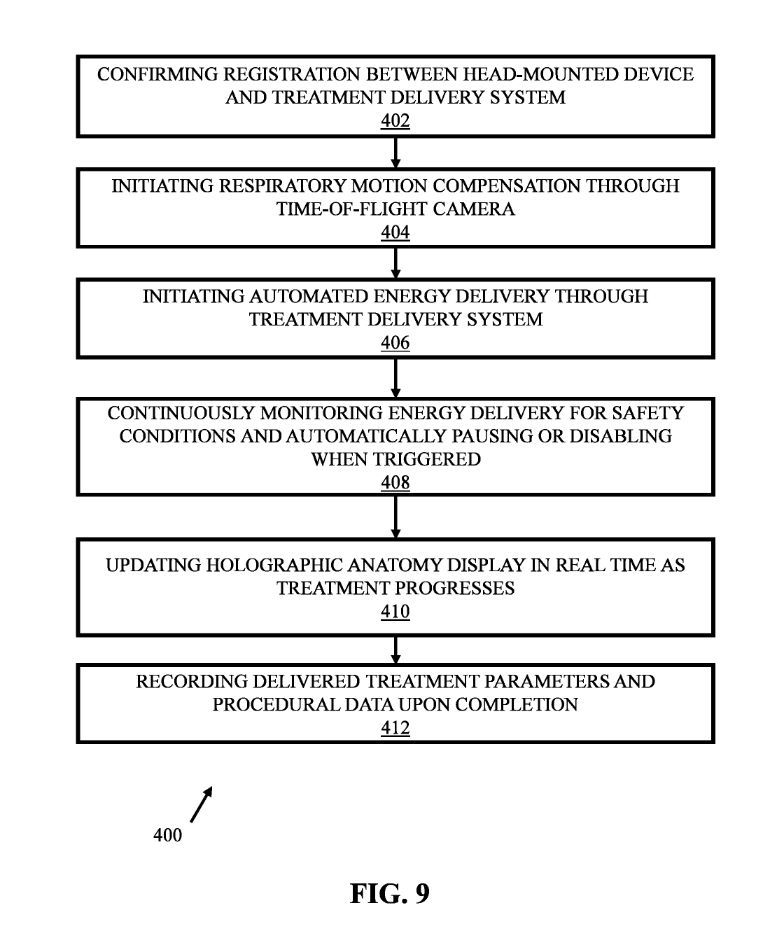

With reference to FIG. 9, a method 400 for delivering non-contacting energy ablation under holographic augmented reality guidance using the system 100 can include the following steps. Method 400 can include a step 402 of confirming registration between the head-mounted device 110 and the treatment delivery system 106 through the registration tracking system 104 and verifying alignment of the treatment head 114 with the planned treatment zone through the holographic display of the augmented reality visualization system 102 before initiating energy delivery, as illustrated in FIG. 9. Method 400 can include a step 404 of optionally initiating respiratory motion compensation through the time-of-flight camera 122 mounted on the treatment head 114, which generates a continuous respiratory phase signal from real-time measurement of chest wall or abdominal surface displacement, and using the respiratory phase signal together with the target positions marked at the breathing extremes during step 312 above to interpolate the current target position throughout the breathing cycle and direct the robotic arm 112 to continuously adjust the position of the treatment head 114 to follow the moving target, or to gate energy delivery to a defined respiratory phase window.

Method 400 can include a step 406 of initiating automated treatment delivery through the treatment delivery system 106, wherein the robotic arm 112 dynamically and continuously moves the treatment head 114 through the planned treatment pattern without requiring continuous manual input from the physician, as illustrated in FIG. 9. Method 400 can include a step 408 of continuously monitoring treatment delivery through the safety interlock logic, including monitoring for protected structure overlap between the active treatment zone and any marked protected structures, monitoring for motion detection through the inertial measurement unit 120, and monitoring physician gaze tracking status through the head-mounted device 110, with the system 100 automatically pausing or disabling energy delivery when any safety condition is triggered, as illustrated in FIG. 9. Method 400 can include a step 410 of updating the holographic display of the anatomy through the augmented reality visualization system 102 in real time as treatment progresses by decimating or deforming the holographic anatomy mesh relative to the treatment pattern's advance through the planned treatment volume, providing the physician with continuous visual feedback on treatment progress, as illustrated in FIG. 9. Method 400 can include a step 412 of recording the delivered treatment parameters, planned therapeutic volumes, robotic arm 112 positioning data, and procedural data through the data storage capabilities of the registration tracking system 104 upon completion of the treatment pattern, enabling retrospective review and post-procedure assessment of treatment zone coverage.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A system for guiding a medical procedure, comprising:
an augmented reality visualization system configured to display a holographic representation;
a treatment delivery system including a robotic linkage and a therapeutic delivery tool selected from the group consisting of: a non-contacting energy delivery device-, a percutaneous device, an ablation device, and a drug delivery device; and
a registration tracking system including a tracking sensor configured to establish a common coordinate system shared by the augmented reality visualization system and the treatment delivery system;
wherein the registration tracking system is configured to maintain continuous spatial alignment between the augmented reality visualization system and the treatment delivery system throughout localization, planning, and treatment phases of the medical procedure; and
wherein the augmented reality visualization system is configured to display a holographic representation of a planned trajectory or therapeutic delivery zone of the therapeutic delivery tool relative to a tracked position and orientation of the therapeutic delivery tool through the augmented reality visualization system.

2. The system of claim 1, wherein the therapeutic delivery tool is a focused ultrasound transducer configured to deliver high intensity focused ultrasound energy or histotripsy energy to a target tissue without contacting the target tissue.

3. The system of claim 1, wherein the treatment delivery system further comprises a diagnostic ultrasound probe integrated with the therapeutic delivery tool.

4. The system of claim 3, wherein an imaging system is configured to acquire real-time diagnostic ultrasound imaging data from the diagnostic ultrasound probe during the localization, planning, and treatment phases of the medical procedure.

5. The system of claim 3, wherein the augmented reality visualization system is further configured to display a holographic representation of an ultrasound beam of the diagnostic ultrasound probe as a flashlight relative to a tracked position and orientation of the diagnostic ultrasound probe through the augmented reality visualization system.

6. The system of claim 1, wherein the tracking sensor comprises at least one of an optical tracking marker, an electromagnetic sensor, an infrared sensor, or an acoustic sensor, wherein the tracking sensor is mounted on the robotic linkage, the therapeutic delivery tool, or both.

7. The system of claim 6, wherein the registration tracking system detects the tracking sensor through a camera of the augmented reality visualization system or through an external tracking system in communication with the augmented reality visualization system to establish the common coordinate system.

8. The system of claim 1, wherein the tracking sensor is mounted on the treatment delivery system, and wherein the registration tracking system is configured to determine a position and orientation of the therapeutic delivery tool in a coordinate system of the treatment delivery.

9. The system of claim 1, further comprising an imaging system and the imaging system is further configured to receive pre-operative imaging data.

10. The system of claim 9, wherein the registration tracking system is configured to register the pre-operative tomographic imaging data to a patient coordinate space.

11. The system of claim 1, wherein the registration tracking system further comprises an additional tracking sensor mounted on or near a patient, and wherein the registration tracking system is configured to detect patient movement relative to the treatment delivery system through the additional tracking sensor and update spatial alignment between the augmented reality visualization system and a patient coordinate space in response to the detected patient movement.

12. The system of claim 1, wherein the augmented reality visualization system is further configured to enable marking of one or more protected anatomical structures associated with the patient, and wherein the registration tracking system is configured to monitor continuously for overlap between an active or planned energy delivery zone of the therapeutic delivery tool, and to perform at least one of highlighting the overlapping protected structure through the augmented reality visualization system, displaying a warning to a user of the guided medical procedure, or automatically disabling energy delivery by the treatment delivery system when overlap is detected.

13. The system of claim 1, further comprising an inertial measurement unit mounted on the robotic linkage and configured to detect changes in position and rotation of the robotic linkage, wherein the registration tracking system is configured to trigger at least one of an automatic re-registration between the augmented reality visualization system and the treatment delivery system or a disabling of energy delivery by the treatment delivery system when the inertial measurement unit detects motion exceeding a defined threshold during the treatment phase of the medical procedure.

14. The system of claim 1, wherein
the registration tracking system is further configured to record procedural data throughout the medical procedure, wherein the procedural data comprises at least one of: pre-operative planning data, real-time imaging data with associated position and orientation data from the tracking sensor, robotic linkage positioning data, energy delivery parameters, and planned therapeutic volumes defined as target contours plus margins stamped and saved in the common coordinate system.

15. A method for localizing a target tissue under holographic augmented reality guidance using the system of claim 1, comprising:
establishing registration between the augmented reality visualization system and the treatment delivery system through the registration tracking system to define the common coordinate system; positioning the therapeutic delivery tool using the robotic linkage to locate the target tissue; and
stamping and saving a position and orientation of the therapeutic delivery tool in the common coordinate system through the registration tracking system,
after one or more iterations of positioning, stamping, and saving, acquiring the target tissue with the therapeutic delivery tool.

16. The method of claim 15, further comprising displaying a holographic representation of a planned trajectory or therapeutic delivery zone of the therapeutic delivery tool relative to the tracked position and orientation of the therapeutic delivery tool through the augmented reality visualization system upon establishing registration.

17. The method of claim 15, further comprising registering pre-operative tomographic imaging data from an imaging system to a patient coordinate space, and displaying three-dimensional holographic anatomical structures overlaid on the patient through the augmented reality visualization system.

18. A method for planning a non-contacting energy delivery procedure under holographic augmented reality guidance using the system of claim 1, comprising:
receiving a stamped position and orientation of the therapeutic delivery tool from the registration tracking system;
directing the robotic linkage to move the therapeutic delivery tool away from a patient to permit preparation of the therapeutic delivery tool;
returning the therapeutic delivery tool to the stamped position; and
defining one or more planned therapeutic volumes relative to a target tissue through the augmented reality visualization system.

19. The method of claim 18, wherein returning the therapeutic delivery tool to the stamped position comprises displaying an offset between a current position of the therapeutic delivery tool and the stamped position through the augmented reality visualization system to guide accurate repositioning.

20. A method for delivering non-contacting therapeutic energy under holographic augmented reality guidance using the system of claim 1, comprising:
confirming registration between the augmented reality visualization system and the treatment delivery system through the registration tracking system;
initiating automated energy delivery through the treatment delivery system wherein the robotic linkage moves the therapeutic delivery tool through a planned energy delivery pattern; and
continuously monitoring energy delivery to confirm completion of the planned energy delivery pattern and to detect one or more safety conditions, and automatically pausing or disabling energy delivery when a safety condition is triggered.
